(19) United States
(12) Patent Application Publication
(10) Pub. No.: US 2009/0216294 A1
(43) Pub. Date: Aug. 27, 2009

(54) SELF-CONTAINED ELECTRONIC MUSCULOSKELETAL STIMULATION APPARATUS AND METHOD OF USE

(75) Inventors: Donald P. EWING, Miami, FL (US); Felix Clarence QUINTANAR, Boca Raton, FL (US); Richard LENSER, Miami, FL (US)

Correspondence Address: CHRISTOPHER & WEISBERG, P.A., 200 EAST LAS OLAS BOULEVARD, SUITE 2040, FORT LAUDERDALE, FL, 33301, US

(73) Assignee: Donald P. EWING, Miami, FL (US)

(21) Appl. No.: 12/345,124

(22) Filed: Dec. 29, 2008

Related U.S. Application Data
(62) Division of application No. 10/777,498, filed on Feb. 12, 2004.

(51) Int. Cl. A61N 1/36

(52) U.S. Cl. 607/46

(57) ABSTRACT

The present invention provides a self-contained electronic musculoskeletal stimulation apparatus that is a battery operated device that applies electronic stimulation to a human with a pre-programmed treatment stimulation protocol to introduce pain relieving electronic stimulation to the body for immediate, symptomatic relief of minor, chronic and acute musculoskeletal aches and pains and mild muscle tension. This invention also provides a method of using a self-contained electronic musculoskeletal stimulation apparatus whereby pain relieving electronic stimulation is applied to the body on predetermined, sequential stimulation points with electronic stimulation being activated at each consecutive stimulation point. Further, this invention provides a method of applying pain relieving electronic stimulation to a body using a self-contained reusable electronic musculoskeletal stimulation bandage with a preprogrammed treatment stimulation protocol. Also provided is a stimulation apparatus that transmits apparatus and patient information by a wireless signal, so the number of times the apparatus was used and intensity level for each use of the apparatus can be determined.

### FIELD OF INVENTION

This invention relates generally to the field of electronic musculoskeletal stimulation apparatus and methods of treating a body with such an apparatus. Specifically, the invention relates to a self-contained electronic musculoskeletal stimulation apparatus that is a battery operated device that applies electronic stimulation to a human with a pre-programmed treatment stimulation protocol to introduce pain relieving electronic stimulation to the body for immediate, symptomatic relief of minor, chronic and acute musculoskeletal aches and pains and mild muscle tension. This invention also relates to a method of using a self-contained electronic musculoskeletal stimulation apparatus whereby pain relieving electronic stimulation is applied to the body on predetermined, sequential stimulation points with electronic stimulation being activated at each consecutive stimulation point.

### BACKGROUND OF THE INVENTION

This invention relates to relief from the pain of minor, chronic, and acute musculoskeletal aches and pains and mild muscle tension associated with stress and other ailments. Pain is typically treated with ingested medications, such as anti-inflammatory and narcotic agents, which can affect a body's motor coordination and/or alter the brain's capacity to function. Alternatively, electrical stimulation of the body can reduce or eliminate pain, prevent or reduce muscle atrophy, increase blood flow to muscles, and increase range of motion and muscle strength. Therefore, electrical stimulation can mask pain without the negative effects caused by ingested pain medication. Electronic stimulation is a safe, non-invasive drug-free method of pain management.

There are several categories of electronic stimulation systems. Some systems which are designed to relieve pain are electronic muscle stimulation systems (“EMS”), transcutaneous electrical neural stimulation systems (“TENS”) and a combination of the two, neuromuscular electronic stimulators (“NMES”). EMS systems are designed to stimulate muscle trigger points and activate inhibitory nerve controls to suppress pain. TENS systems are designed to stimulate large sensory nerve endings to help decrease pain by masking the smaller adjacent pain nerves. EMS stimulation is characterized by a low volt stimulation targeted to stimulate motor nerves to cause a muscle contraction. TENS stimulation is characterized by biphasic, low volt current and selectable parameters of pulse rate and pulse width. Stimulation of both nerve and muscle by the NMES systems triggers the chemical release of beta-endorphin, a natural opioid, with potent analgesic effects.

Typically, the brain controls and causes muscles to relax or contract by sending nerve impulses to the muscle. EMS uses electrical current to stimulate nerve axons within certain muscles and causes them to contract. The muscle contractions can relieve pain in sore muscles, prevent or reduce muscle atrophy, increase blood flow to muscles, and increase range of motion and muscle strength. EMS is commonly used after orthopedic surgery, joint replacement, to reduce muscle spasms, to increase strength of the muscles, to prevent or reduce disuse atrophy, to prevent pain from arthritis or stress, and generally to help heal and reduce pain from injuries to joints and tendons.

A device as described in United States Application Publication No. 20020019652 to Da Silva et al. describes a two part transcutaneous electrical nerve stimulator (“TENS”) bandage. The device combines a sterile bandage with a TENS device for use in covering a wound and providing electrical stimulation to promote healing and block pain. The device is designed so that the bottom portion of the device is a one time use sterile bandage that is attachable to a top portion containing the electronics module. The top portion can be reused multiple times. The bandage is designed to be worn over a wound until the wound heals. This invention does not provide for a bandage that would cause the muscle to contract and provide the desired pain relief. Additionally, this bandage is designed to block pain from an open wound and would not effectively block pain from muscles or joints.

A device as described in U.S. Pat. No. 5,423,874 to D'Alerta describes an oval patch with a curved profile for applying pain reducing electrical energy to the body. The device is comprised of four layers: a circuit layer which supports an electronic circuit, a double sided adhesive layer, a top layer which seals the circuit layer from moisture and a backing layer. A cathode and anode are disposed in apertures of the adhesive layer and make electrical contact with respective pins of the electronic circuit in the circuit layer. The current and voltage are in phase and are sustained at a level greater than zero for a short period of time, in repeated pulses or bursts. A 50% duty cycle waveform is created comprising a series of spaced 50 volt pulses with a period of 0.25 to 2 seconds. The device of this invention is inadequate because of the lower level of voltage output by the device and the complicated construction of the bandage made up of several layers. Additionally, the length of the period used by this invention is too long to obtain the desired pain relieving effect of the current invention.

U.S. Pat. No. 5,183,041 to Toriu et al. describes a transcutaneous electric nerve stimulator having a plurality of treatment modes. The TENS stimulator produces a low-frequency pulse of a frequency corresponding to a selected treatment mode. The device has a plurality of indicators in association with the respective treatment modes such that one of the indicators corresponding to the selected treatment mode is caused to blink in synchronism with the produced low-frequency pulse. The TENS stimulator; however, only produces a lower level of voltage and uses a switch for each treatment mode.

U.S. Pat. No. 5,562,718 to Palermo describes an electronic neuromuscular stimulation device that is operated by a computerized electronic control unit that includes at least two output channels to which are connected a corresponding set of electrode output cables. The unit also includes controls, indicators, and circuitry that produce nerve stimulation pulses. The unit can produce pulse trains and pulse train patterns, including, sequential patterns, delayed overlapping patterns, triple-phase overlapping patterns, reciprocal pulse trains, and delayed sequenced sprint interval patterns. This unit uses electrode cables to connect the device to the electrodes. The patent also discloses appropriate electrode placement on the human body, specifically the agonist and antagonist placement for activation of certain muscle groups. The device is inadequate and bulky, because it requires cables to connect the electrodes to the control unit. Further, the device primarily uses pulse trains or patterns to gently twitch the muscle and is designed to take advantage of neurological enhancement.

U.S. Pat. No. 4,232,680 to Hudleson et al. describes a method and apparatus for transcutaneous electrical nerve stimulation. The apparatus functions so that the area of body operably interposed between the pads will be treated by the squarewave signal having a predetermined constant current level, which is independent of the operative resistance of the body section between the pads. The apparatus includes a first circuit operatively coupled to a source of electrical energy for generating a squarewave output signal. At least two pads are provided for being placed on the skin adjacent to the body sections of the patient to be treated. A second circuit is provided having an input coupled to the pads. The second circuit amplifies the squarewave signal from the first circuit so as to deliver at the outputs thereof a predetermined constant current squarewave output signal representative of the input signal. The device contains an indicator signal for indicating when the constant current output is equal to the prescribed current setting. The system uses paired pads connected to the device by cables and is therefore bulky and inadequate. Further, this system is designed specifically to produce a constant current irrespective of the resistant forces of the body.

One of the problems associated with current electronic muscle stimulation systems is that they are bulky and require leads or cables which connect the electrodes to the electrical current generator and controls. There is a need in the art for a self-contained NMES system where the control circuit and electrodes are within the same housing, and the housing functions like a bandage, so that the device is easily portable and fits close to the body. There is also a need for a user friendly device that can be applied by a patient without the need to seek assistance from a medical professional.

Additionally, there is a need for a method of applying pain relieving electronic stimulation to a body by the use of predetermined, sequential stimulation points leading up to the point of pain with electronic stimulation being activated at each consecutive stimulation point. The currently available devices treat pain by placement of the device on the specific point of pain. This method of treatment can bypass the actual source of the pain rendering the treatment ineffective. Therefore, alternate methods of applying electronic stimulation to the body are needed.

Consequently, there is a need in the art for a self-contained reusable electronic musculoskeletal stimulation apparatus containing a control circuit connected directly to electrodes, with the electrodes and control circuit contained in the same housing, where the housing functions like a bandage, so that the device is easily portable and fits close to the body. There is also a need in the art for a method of applying pain relieving electronic stimulation to a body on predetermined, sequential stimulation points leading up to the point of pain with electronic stimulation being activated at each consecutive stimulation point. It is also desirable to have a method of applying pain relieving electronic stimulation to a body using a self-contained reusable electronic musculoskeletal stimulation apparatus with a preprogrammed treatment stimulation protocol.

### SUMMARY OF THE INVENTION

The present invention solves significant problems in the art by providing a self-contained electronic musculoskeletal stimulation apparatus that is a battery operated device that applies electronic stimulation to a human with a pre-programmed treatment stimulation protocol to introduce pain relieving electronic stimulation to the body for immediate, symptomatic relief of minor, chronic and acute musculoskeletal aches and pains and mild muscle tension associated with stress and other ailments. The stimulation apparatus consists of electrodes attached directly to a control circuit, which are housed in two layers of polyvinylchloride (“PVC”), or other suitable housing material, along with insulating foam. The flexible housing is water resistant and the apparatus is designed to be re-used by the patient. The stimulation apparatus can be various shapes in order to resemble a bandage and fit the curves of a human.

The above and other objects of the invention are achieved in the embodiments described herein by incorporating two operational buttons into the stimulation device. The first button, hereinafter called the power button, is programmed to power the device on or off and control the intensity or amplitude of the electronic stimulation. The intensity ranges from low, medium or high and the selection of the particular intensity is reflected by an indicator, typically a light emitting diode (“LED”) display, or a graph incorporated onto the circuit, such as a liquid crystal display (“LCD”) display. Each intensity range has a corresponding indicator which lights up to indicate the activation or selection of a particular intensity. The second button, hereinafter called the treatment button, is utilized to activate a predetermined electronic stimulation after the device has been powered and set to the desired setting or intensity. The device remains in the off position and only emits an electronic stimulation while the treatment button is pressed. The aforementioned indicators also acknowledge the activation of the system. While the treatment button is depressed and the treatment activated, one of the indicators will flash identifying the particular intensity currently being administered. When the user ceases pressing the treatment button, the electronic stimulation will stop. All resistors, diodes and hardware that are utilized to create the electronic stimulation or frequency are controlled by a microprocessor and programming to allow for the multiple intensity and frequency or pulse variations.

The present invention also uses various treatment stimulation protocols. The treatment stimulation protocol is transmitted to predetermined stimulation points on the body by replaceable electro-gel pads covering the electrodes on the backside of the stimulation apparatus. Treatment typically begins at predetermined stimulation points furthest from the area of pain, but in the same general area of the body. For example, for foot pain in the ball of the foot, treatment begins at stimulation points on the ankle and continues to the ball of the foot. The stimulation apparatus is also capable of being used to provide stimulation on the area of pain, directly over the area from which pain is emanating, and encircling the area of pain. The apparatus delivers a 1 to 4 second duration treatment and the voltage intensity is adjustable between three levels of intensity. The present invention is also capable of transmitting apparatus and patient information by a wireless signal, so the number of times the apparatus was used and intensity level for each use of the apparatus can be determined by the manufacturer or clinician.

In an alternate embodiment, the housing of the self-contained electronic musculoskeletal stimulation apparatus is made from a thermoplastic material, such as a polycarbonate resin. One embodiment uses a polycarbonate resin sold under the trademark LEXAN® of General Electric Company. A thermoplastic material provides an extremely durable housing and therefore, the apparatus will still be in good condition when the battery runs out of power. Thus, the apparatus made of thermoplastic material is designed to have a lid, which opens to reveal the electronics. The battery, among other portions of the electronics, is replaceable. Additionally, the apparatus made of thermoplastic material is also designed to be slidably secured into a case made of thermoplastic material. The back of the apparatus, and thus the electrogel pads, would be protected by the case and the case would allow for easy transport of the apparatus.

In another embodiment, the self-contained electronic musculoskeletal stimulation apparatus contains a sensor to locate pain on the human body. Alternately, the sensor can be used to locate stimulation points on the body. The sensor is a transepithelial potential indicator, which measures frequency across the skin to identify the termination of nerve ends within the body. When the sensor passes over the nerve ends, the sensor can read a change in frequency across the skin and the sensor alerts the patient to notify the patient that it has passed the apparatus over a stimulation point or a possible point of pain.

In yet another embodiment, the self-contained electronic musculoskeletal stimulation apparatus may contain medicine in the gel pad. The apparatus would function to enhance the absorption of the medicine into the skin and muscles through iontophoresis. Iontophoresis devices use a direct or alternating current to introduce ions of soluble salts or other drugs into the body for medical purposes. Iontophoresis has been shown to provide the most rapid resolution to muscular pain when compared with orally administered muscle relaxant and analgesic medications. When the apparatus is used for iontophoresis, typically a much higher frequency of electronic stimulation would be used. The frequency generally used in iontophoresis applications is approximately 4000 hertz.

### BRIEF DESCRIPTION OF THE DRAWINGS

FIG. 1 is a front view of the self-contained electronic stimulation apparatus.

FIG. 2 is the backside of the self-contained electronic stimulation apparatus.

FIG. 3 is a diagram of the back and front view of head and neck pain stimulation points.

FIG. 4 is a diagram of the right profile of head and neck pain stimulation points.

FIG. 5 is a diagram of an oblique view of head and neck pain stimulation points.

FIG. 6 is a diagram of the back and front view of chest, back and arm pain stimulation points.

FIG. 7 is a diagram of the right profile of chest, back and arm pain stimulation points.

FIG. 8 is a diagram of the front view of chest, back and arm pain stimulation points.

FIG. 9 is a diagram of the back and front view of abdomen, low back and forearm pain stimulation points.

FIG. 10 is a diagram of the right profile of abdomen, low back and forearm pain stimulation points.

FIG. 11 is a diagram of the back and front view of buttock, groin, wrist and hand pain stimulation points.

FIG. 12 is a diagram of the right profile of buttock, groin, wrist and hand pain stimulation points.

FIG. 13 is a diagram of the back and front view of hip, thigh and knee pain stimulation points.

FIG. 14 is a diagram of the right profile of hip, thigh and knee pain stimulation points.

FIG. 15 is a diagram of the back and front view of leg and foot pain stimulation points.

FIG. 16 is a diagram of the right profile of leg and foot pain stimulation points.

FIG. 17 is a diagram of the foot and ankle pain stimulation points.

FIG. 18 is a diagram of an overview of stimulation points on the body and the corresponding reference diagram on the stimulation apparatus.

FIG. 19 is a functional diagram of the stimulation apparatus.

FIGS. 20A-F are flowcharts of the stimulation apparatus software.

FIG. 21 is a schematic diagram of the circuit of the stimulation apparatus.

FIGS. 22A-C are graphs of pulse-width modulation techniques.

FIG. 23 is a graph of the discharge pulse waveform.

FIG. 24 is a graph of the total discharge pulses.

FIGS. 25A and B are graphs of various low intensity pulses.

FIGS. 26A and B are graphs of various medium intensity pulses.

FIGS. 27A and B are graphs of various high intensity pulses.

### DETAILED DESCRIPTION OF THE PREFERRED EMBODIMENT

While the invention is susceptible of several embodiments, there is shown in the drawings, specific embodiments thereof, with the understanding that the present disclosure is to be considered as an exemplification of the invention and is not intended to limit the invention to the specific embodiments shown.

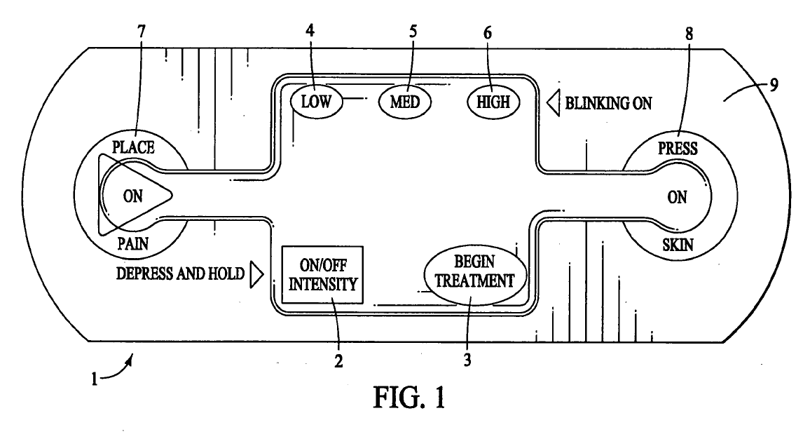

Referring initially to FIG. 1 of the drawings, in which like numerals indicate like elements throughout the several views, a front view of the self-contained electronic stimulation apparatus 1 is shown. The electronic stimulation apparatus 1 is shown in the shape of a rectangular bandage with curved ends. The housing 9 of the apparatus 1 consists of two layers of polyvinylchloride (“PVC”) or other suitable material, such as any flexible, hypoallergenic material or polycarbonate resin. Between the two layers of PVC is an insulation sheet that surrounds the electronic components of the apparatus 1. The insulation sheet is made of foam and functions to protect the hardware and prevent the device from burning the user. A control circuit and electrodes 15 are enclosed in the housing 9. The flexible housing 9 is water resistant and the apparatus 1 is designed to be re-usable by the patient. The housing 9 may be printed on, for example by silk-screening, to label the buttons for ease of use.

The power button 2 controls turning the apparatus 1 on and off and allows the patient to select the intensity of the electronic stimulation. As a safety measure, the apparatus 1 will always default to the off position when a button is not actively being pressed. The intensities range from low 4, medium 5 and high 6. Once the apparatus 1 is powered by a patient pushing the power button 2, the patient may further depress and hold down the power button 2 to choose an intensity level. While the patient depresses and holds the power button 2, the indicator for low 4, medium 5 and high 6 will light up sequentially indicating each particular intensity. Indicators 4, 5, and 6 may be light emitting diodes or some other suitable form of indication display. The indicators 4, 5 or 6 light up to indicate that the patient can select the particular indicated intensity by simply ceasing to depress the power button 2.

Once the patient has selected an intensity level, he or she must attach the apparatus 1 to their body. The patient should press the raised distal-end of the electrode 7, found on the front, left side of apparatus 1, onto one of the following locations: a predetermined stimulation point, the area of the pain site, directly over the area from which pain is emanating, or encircling the area of pain. The raised distal-end of the electrode 7 typically has an arrow on it to denote that it should be placed on the stimulation point or site of pain. The stimulation points are discussed in further detail later. The raised distal-end of the electrode 8, found on the right side of apparatus 1, should also be pressed onto the skin to hold the apparatus 1 in place.

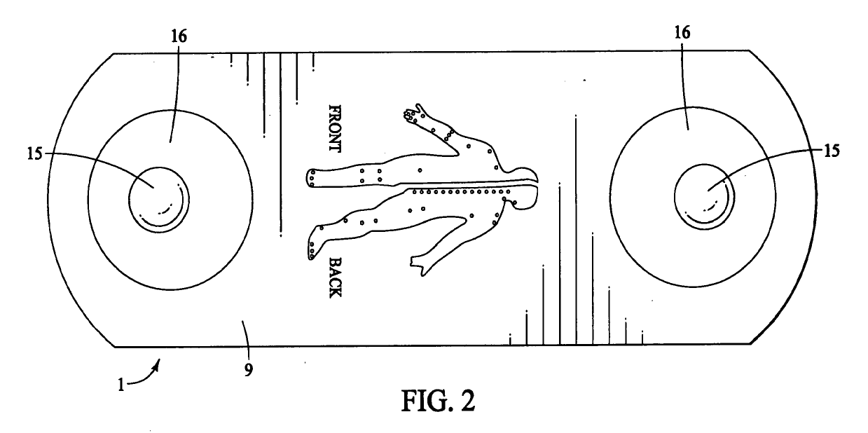

FIG. 2 shows the backside of the self-contained electronic stimulation apparatus. The patient attaches the apparatus 1 onto the body with the backside of the apparatus 1 being in contact with the patient's skin. The apparatus 1 attaches to the body with electrogel pads 16, which cover the proximal-end of the electrode 15. When the patient presses the raised distal-ends of electrodes 7 and 8 onto the skin, the electrogel pads 16 function as adhesive to hold the apparatus 1 in place. The electrogel pads 16 also reduce skin resistance and allow the current to flow between the electrodes 15. The electrogel pads may be composed of hydrogel or a tacky, sticky adhesive material. The electrogel pads 16 are replaceable, so that the apparatus 1 may be reused. When the electrogel pads 16 are composed of hydrogel, the hydrogel can be placed on the apparatus 1 and attached to the patient by utilizing the hydrogel's inherent adhesive properties. Alternatively, the electrogel pads 16 can include a fastening arrangement, such as a snap, which holds the electrogel pad 16 onto the apparatus 1. For example, a fastening arrangement could include a male component of a snap located on the bottom of the apparatus 1, which couples with a female component of the snap incorporated on the backside of the electrogel pad 16. When the snap is manually engaged, the electrogel pad 16 will remain attached to the apparatus 1 until the snap is disengaged. The patient must be sure that both electrogel pads 16 contact the skin when attaching the apparatus 1 to their body. Referring back to FIG. 1, the patient can begin treatment by depressing and holding down the treatment button 3. When the treatment button 3 is depressed, one of the indicators 4, 5, or 6 will blink rapidly to indicate which intensity is currently being used to treat the patient. Therefore, indicator's 4, 5, and 6 will provide the patient with the identification of the intensity being used by the patient and an indication of treatment beginning. Once the patient ceases to depress the treatment button 3, the electronic stimulation will stop and the apparatus 1 will power off.

When the apparatus 1 is first used, the patient should begin intensity levels with the low 4 intensity stimulation. After a patient is accustomed to the apparatus 1, the stimulation intensity should be set as high as possible without causing the patient discomfort. The four second stimulation is automatically determined by the microcontroller and activated by pressing and holding down the treatment button 3. The patient may use less than a four second stimulation by ceasing to hold down the treatment button 3. Upon release of the treatment button 3, the apparatus will power off. The temporary pain-inhibiting effect should commence immediately after stimulation. However, in some cases desensitizing must be carried out for several applications prior to successful pain relief by the apparatus 1.

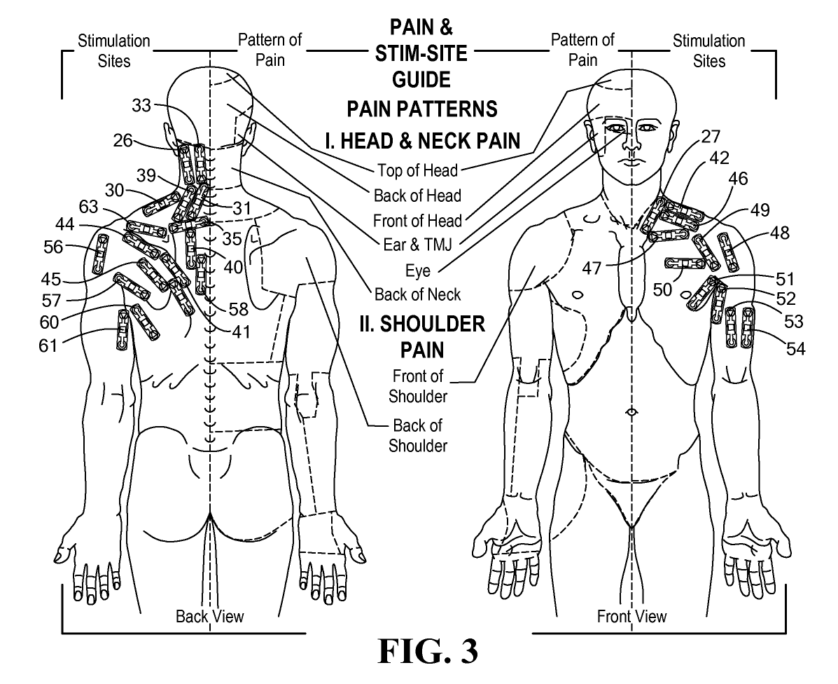

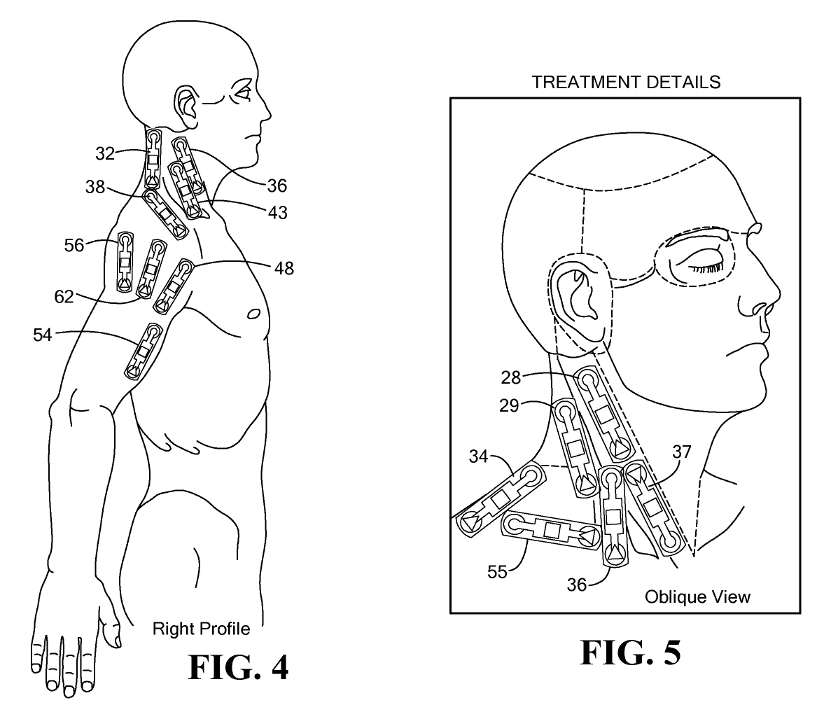

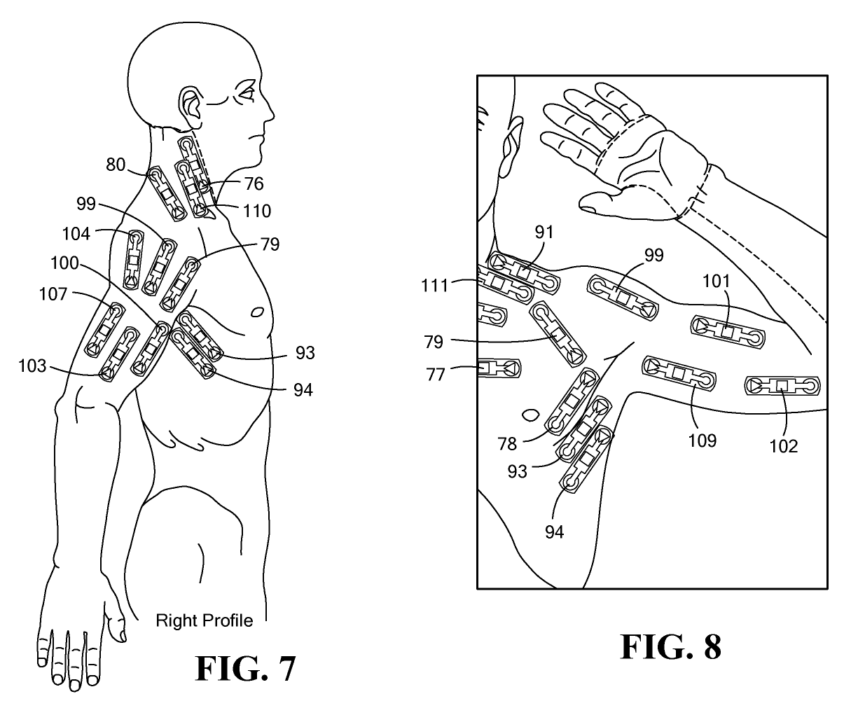

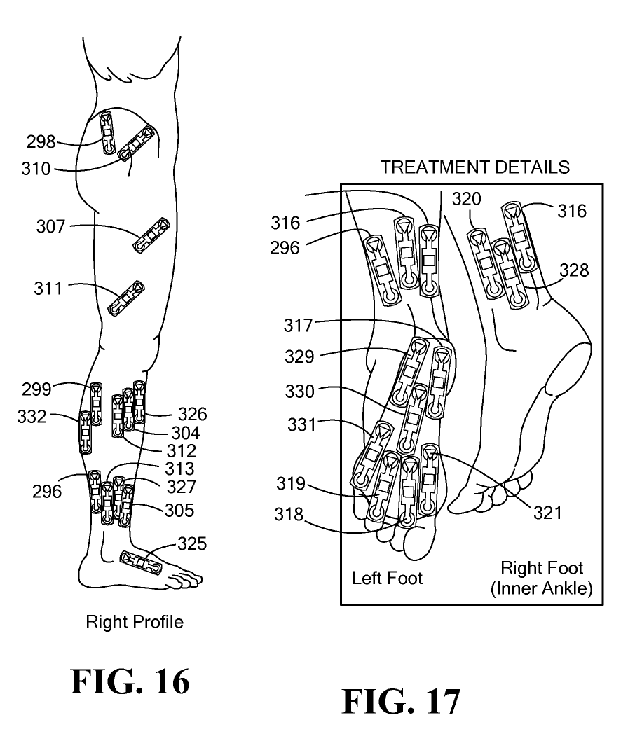

The stimulation apparatus 1 uses a low intensity direct electrical stimulation with preprogrammed stimulation parameters. When the predetermined stimulation points depicted in FIGS. 3-18 are stimulated, they block pain by activation of the larger superficial sensory nerves adjacent to the source of pain. These activated sensory nerves are preferentially processed by the nervous system, thus activation of these nerves masks information from smaller pain nerves. The stimulation points identified in FIGS. 3-18 are essentially muscle trigger points. Stimulation with apparatus 1 of the muscle trigger points will elicit a focal contraction of the muscle positioned under the apparatus 1. The elicited contraction of these muscle trigger points activates inhibitory controls suppressing both local and remote sources of pain. Additionally, use of the apparatus 1 to stimulate either large sensory fibers or muscle causes the local release of endogenous chemicals including the opioid beta endorphin which has a potent analgesic effect.

One way to use the stimulation apparatus 1 is to apply pain relieving electronic stimulation to a body by the use of predetermined, sequential stimulation points with stimulation being activated at each designated stimulation point. For effective relief of sharp or prolonged aches and pains, the treatment should begin with the apparatus 1 placed over stimulation points furthest from the area of pain but in the same general area of the body. The apparatus 1 user simply has to locate the source of their pain on the body according to FIGS. 3-18 to identify the specific array of stimulation sites, which will relieve that area of pain. The order the sites are simulated should begin furthest from the exact location of pain, with the last stimulation positioning the positive electrode of the apparatus 1 directly over the most intense source of pain. For example, FIGS. 3, 4 and 5 show various stimulation points for relief from head and neck pain. For pain in the top of the head, a patient attaches the apparatus 1 by pressing the raised distal-end of the electrode 7 onto stimulation point 26 and the distal-end of the electrode 8 onto a respective area of the body to hold the apparatus 1 in place. As such, the electrogel pads 16, covering the proximal-ends of the electrodes 15 and found on the backside of the apparatus 1, will be in contact with the skin. Next, the patient selects an intensity of stimulation and activates the stimulation. After treatment of stimulation point 26, the patient removes the apparatus 1 from his or her body and moves the apparatus 1 to the next stimulation point 28, which is located on the front of the body at the lower neck. After stimulating point 28, the patient repeats such stimulation on stimulation point 29.

After all of the stimulation points leading to the painful area have been stimulated, the patient should move the afflicted body part to determine if they still feel pain. If the patient still feels pain, he or she should locate the exact origin of the point of pain and place the raised distal-end of the electrode 7 of the apparatus 1 directly on the point of pain and the distal-end of the electrode 8 onto the respective area of the body to hold the apparatus 1 in place. The electrogel pads 16 that cover the proximal-ends of both electrodes 15 should be in contact with the skin. Then the patient should activate the stimulation. If pain persists, the patient should again try to determine the exact origin of the point of pain and place the raised distal-end of the electrode 7 of the apparatus 1 directly on the point of pain and the distal-end of the electrode 8 onto the respective area of the body to hold the apparatus 1 in place and repeat stimulation.

For pain in the back of the head, the patient uses the same procedure outlined above and stimulates points 30, 31 and 32 in that order. For pain in the front of the head, the patient uses stimulation points 33, 34 and 27 in that order. For pain in the ear and for TMJ or jaw pain, the patient stimulates points 35 and 36 respectively. For eye pain, the patient stimulates 33, 37 and 38. For pain in the back of the neck, the patient simulates points 26, 39, 40, 41, 42, and 43. Additionally shown in FIGS. 3, 4 and 5 are stimulation points for shoulder pain. For pain in the front of the shoulder, the patient stimulates points 44, 45, 46, 47, 48, 49, 50, 51, 52, 53, 54 and 55 in that order. For pain in the back of the shoulder, the patient stimulates points 56, 57, 58, 59, 60, 61, 62 and 63 respectively.

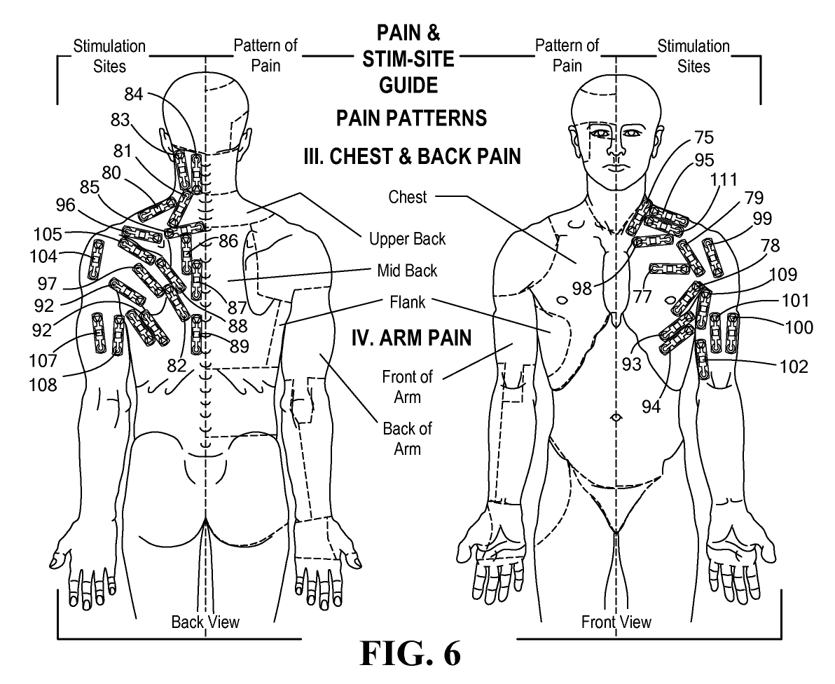

FIGS. 6, 7 and 8 diagram the stimulation points that are used when pain is felt in chest and back or arm. For chest pain, a patient attaches the apparatus 1, specifically the raised distal-end of the electrode 7, at stimulation point 75 on his or her lower neck and the distal-end of the electrode 8 onto the respective area of the body to hold the apparatus 1 in place. The electrogel pads 16, covering the proximal-ends of the electrodes 15 and found on the backside of the apparatus 1, should be in contact with the skin. Next, the patient selects an intensity of stimulation and activates the stimulation. After treatment of stimulation point 75, the patient removes the apparatus 1 from his or her body and moves the apparatus 1 to the next stimulation point 76. After stimulating point 76, the patient repeats such stimulation on stimulation points 77, 78, and 79.

After all of the stimulation points leading to the painful area have been stimulated, the patient should move their chest muscles to determine if they still feel pain. If the patient still feels pain, he or she should locate the exact origin of the point of pain and place the raised distal-end of the electrode 7 of the apparatus 1 directly on the point of pain and the distal-end of the electrode 8 onto the respective area of the body to hold the apparatus 1 in place. Both electrogel pads 16 that cover the proximal-ends of both electrodes 15 should be in contact with the skin. If pain persists, the patient should again try to determine the exact origin of the point of pain and place the raised distal-end of the electrode 7 of the apparatus 1 directly on the point of pain, place the distal-end of the electrode 8 onto the respective area of the body to hold the apparatus 1 in place and repeat stimulation.

For pain in upper back, the patient uses the same procedure outlined above and stimulates points 80, 81, 82, 83, and 84 in that order. For pain in the mid back, the patient stimulates points 85, 86, 87, 88, 89, 90 and 91. For pain in the flank region, the patient stimulates points 92, 93 and 94. FIGS. 6, 7 and 8 also diagram the stimulation points to be used to alleviate pain in the front and back arm. For pain in the front of the arm, the patient stimulates points 96, 97, 98, 99, 100, 101, 102, and 103 respectively. For pain in the back of the arm, the patient stimulates points 104, 105, 106, 107, 108, 109, 110, and 111.

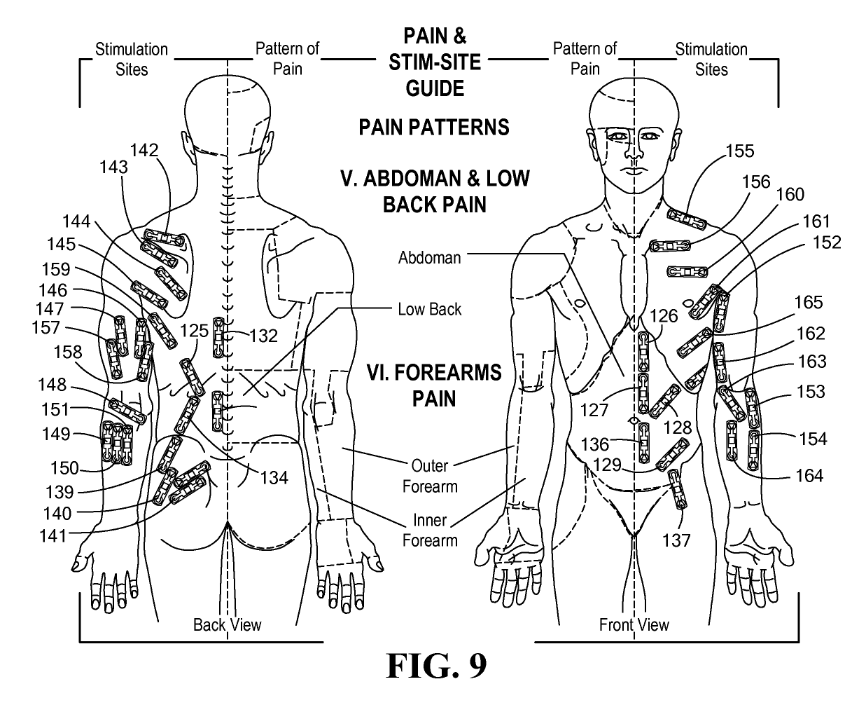

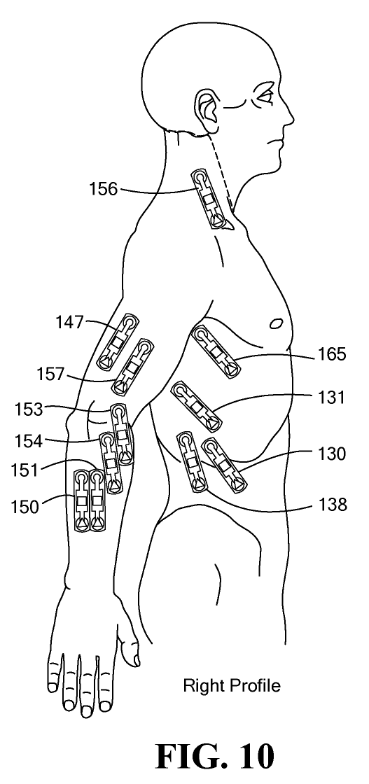

Now referring to FIGS. 9 and 10, when the patient applies the apparatus for abdomen, lower back or forearm pain, the patient should use the noted corresponding stimulation points. For example, for abdomen pain, the patient begins with stimulation point 125. After treatment of stimulation point 125, the patient removes the apparatus 1 from his or her body and moves the apparatus 1 to the next stimulation point 126. After stimulating point 126, the patient repeats such stimulation on stimulation point 127. Next, the patient would stimulate the abdomen stimulation points 128, 129, 130, and 131. Again, after all of the stimulation points leading to the painful area have been stimulated, the patient should move their abdomen muscles to determine if they still feel pain. If the patient still feels pain, he or she should locate the exact origin of the point of pain and place the raised distal-end of the electrode 7 of the apparatus 1 directly on the point of pain and the distal-end of electrode 8 onto the respective area of the body to hold the apparatus 1 in place. Both of the electrogel pads 16 that cover the proximal-ends of both electrodes 15 should be in contact with the skin. If pain persists, the patient should again try to determine the exact origin of the point of pain and place the raised distal-end of the electrode 7 of the apparatus 1 directly on the point of pain, place the distal-end of electrode 8 onto the respective area of the body to hold the apparatus 1 in place, and activate stimulation. For low back pain, the patient would stimulate points 132, 133, 134, 135, 136, 137, 138, 139, 140, and 141 sequentially.

FIGS. 9 and 10 also show stimulation points to be used for forearm pain. For outer forearm pain, the patient should stimulate points 142, 143, 144, 145, 146, 147, 148, 149, 150, 151, 152, 153, 154, 155, 156, 157, and 158 in that order. After all of the stimulation points leading to the painful area have been stimulated, the patient should move their outer forearm muscles to determine if they still feel pain. If the patient still feels pain, he or she should locate the exact origin of the point of pain and stimulate that point. For inner forearm pain, the patient should stimulate points 159, 160, 161, 162, 163, 164, and 165 sequentially. Again, after all of the stimulation points leading to the painful area have been stimulated, the patient should move their inner forearm muscles to determine if they still feel pain. If the patient still feels pain, he or she should locate the exact origin of the point of pain and stimulate that point.

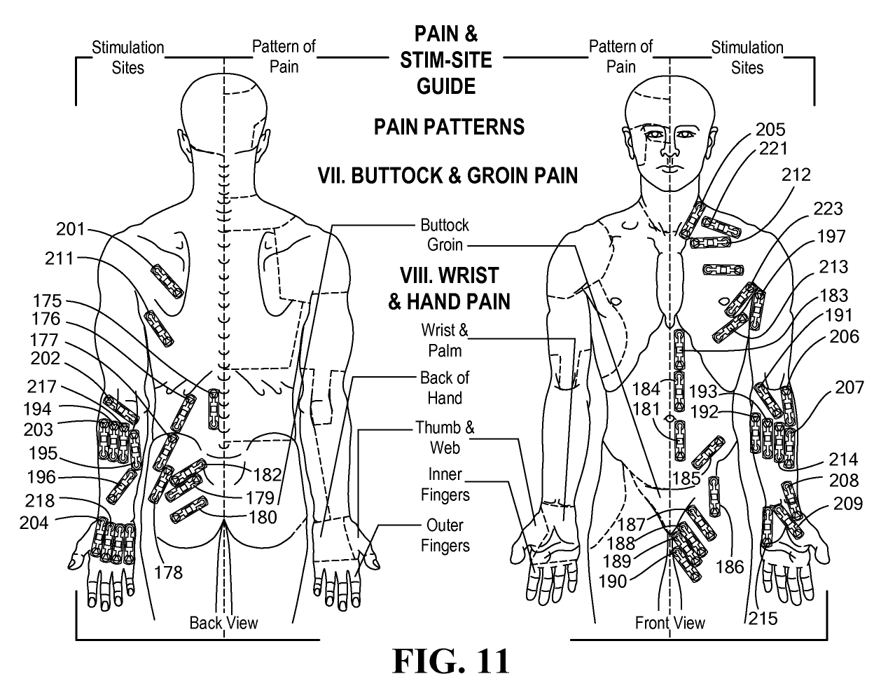

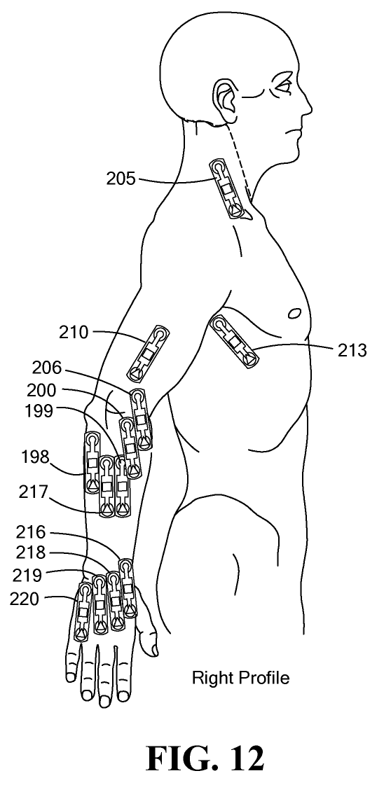

FIGS. 11 and 12 are diagrams of the various stimulation points to be used when the patient feels buttock, groin, wrist and hand pain. For buttock pain, the patient should stimulate points 175, 176, 177, 178, 179, 180, and 181 sequentially. After all of the stimulation points leading to the painful area have been stimulated, the patient should move their muscles to determine if they still feel pain. If the patient still feels pain, he or she should locate the exact origin of the point of pain and stimulate that point. For groin pain, the patient should stimulate points 182, 183, 184, 185, 186, 187, 188, 189, and 190 sequentially. After all of the stimulation points leading to the painful area have been stimulated, the patient should move their muscles to determine if they still feel pain. If the patient still feels pain, he or she should locate the exact origin of the point of pain and stimulate that point.

Referring again to FIGS. 11 and 12, for wrist and palm pain, the patient should stimulate points 191, 192, and 193 sequentially. If the patient still feels pain after stimulation, he or she should locate the exact origin of the point of pain and stimulate that point. For back of hand pain, the patient should stimulate points 194, 195, 196, 197, 198, 199, and 200 sequentially. If the patient still feels pain after stimulation of the back of the hand stimulation points, he or she should locate the exact origin of the point of pain and stimulate that point. For thumb and web pain, the patient should stimulate points 201, 202, 203, 204, 205, 206, 207, 208, 209 and 210 sequentially. After all of the stimulation points leading to the painful area have been stimulated, the patient should move their thumb or web muscles to determine if they still feel pain. If the patient still feels pain in their thumb or web muscles, he or she should locate the exact origin of the point of pain and stimulate that point. For inner finger pain, the patient should stimulate points 211, 212, 213, 214, 215, and 216 sequentially. If the patient still feels pain after stimulation, he or she should locate the exact origin of the point of pain and stimulate that point. For outer finger pain, the patient should stimulate points 217, 218, 219, 220, 221, 222, and 223 in that order. If the patient still feels pain after stimulation for outer finger pain, he or she should locate the exact origin of the point of pain and stimulate that point.

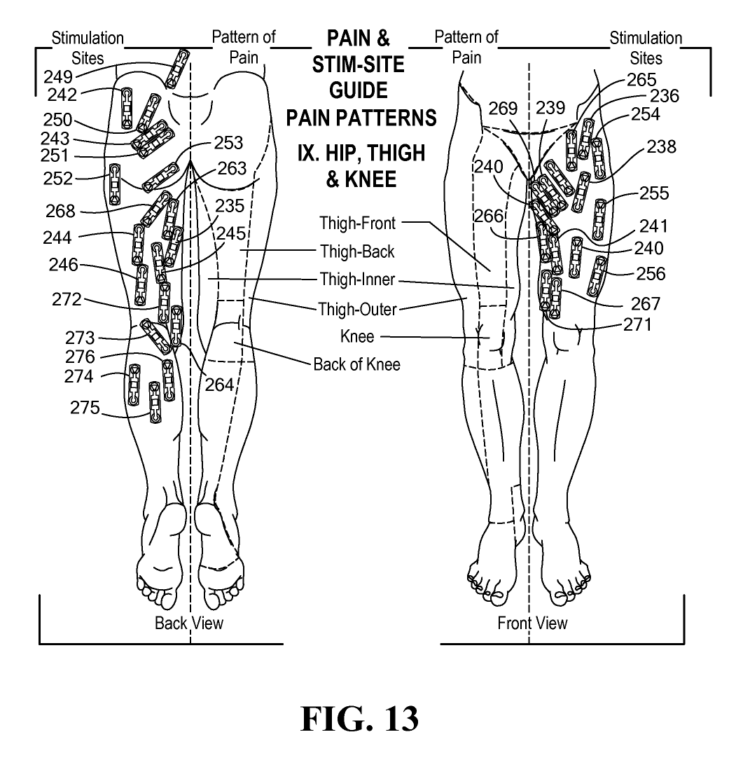

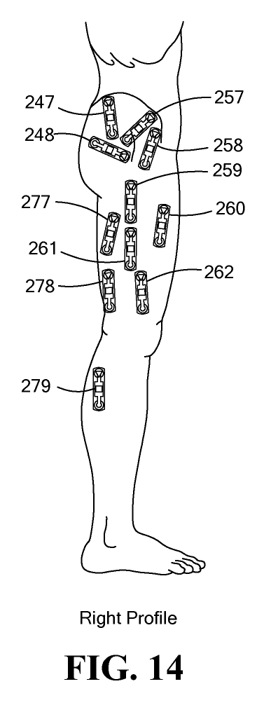

FIGS. 13 and 14 diagram stimulation points for pain in the hip, thigh and knee. For pain in the front thigh, the patient should sequentially stimulate points 236, 237, 238, 239, 240, and 241. If the patient still feels pain after stimulating all of the stimulation points on the front of the thigh, he or she should locate the exact origin of the point of pain and stimulate that point. For pain in the back of the thigh, the patent should stimulate points 242, 243, 244, 245, 246, 247 and 248. If the patient still feels pain after stimulating all of the stimulation points on the back of the thigh, he or she should locate the exact origin of the point of pain and stimulate that point. For pain in the outer thigh, the patient should stimulate points 249, 250, 251, 252, 253, 254, 255, 256, 257, 258, 259, 260, 261, and 262. Again, if the patient continues to feel pain after stimulating all of the stimulation points on the outer thigh, he or she should locate the exact origin of the point of pain and stimulate that point. For pain in the inner thigh, the patient should stimulate points 263, 264, 265, 266, and 267 sequentially. If the patient continues to feel pain after stimulating all of the stimulation points on the inner thigh, he or she should locate the exact origin of the point of pain and stimulate that point. For knee pain, the patient should sequentially stimulate points 268, 269, 270 and 271. If the patient continues to feel knee pain after stimulating all of the stimulation points, he or she should locate the exact origin of the point of pain and stimulate that point. For pain in the back of the knee, the patient should stimulate points 272, 273, 274, 275, 276, 277, 278, and 279 sequentially. If the patient continues to feel pain in the back of the knee after stimulating all of the stimulation points, he or she should locate the exact origin of the point of pain and stimulate that point.

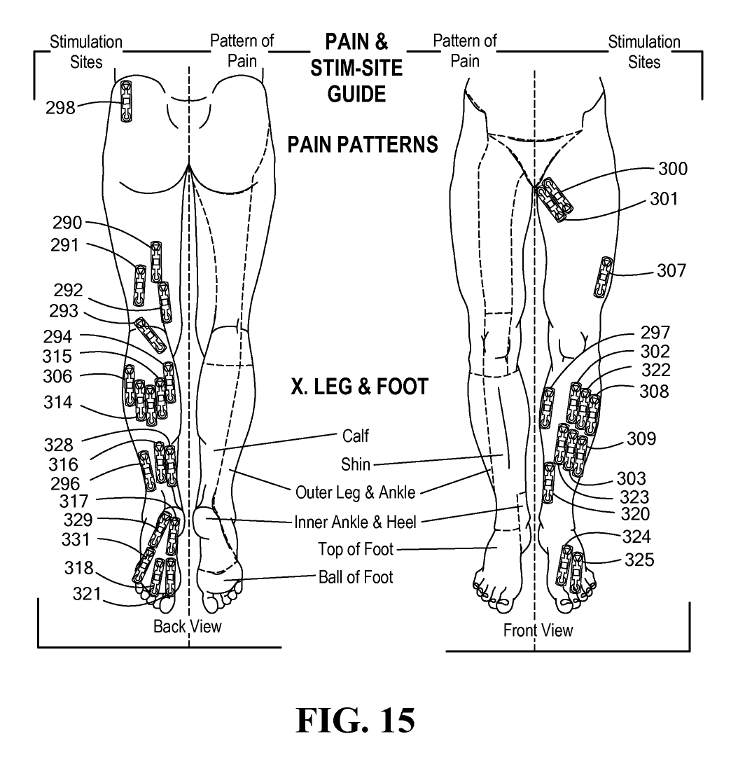

FIGS. 15, 16 and 17 diagram stimulation points to be used with leg and foot pain. For pain in the calf muscle, the patient should sequentially stimulate points 290, 291, 292, 293, 294, 295, 296, 297, 298 and 299. If the patient continues to feel pain in the calf muscle after stimulating all of the stimulation points, he or she should locate the exact origin of the point of pain and stimulate that point. For pain in the shin, the patient should stimulate points 300, 301, 302, 303, 304, and 305. If the patient still feels pain after stimulating all of the stimulation points for the shin, he or she should locate the exact origin of the point of pain and stimulate that point. For pain in the outer leg and ankle, the patient should stimulate points 306, 307, 308, 309, 310, 311, 312 and 313. If the patient still feels pain after stimulating all of the stimulation points for outer leg and ankle, he or she should locate the exact origin of the point of pain and stimulate that point. For pain in the inner ankle and heel, the patient should stimulate points 314, 315, 316, 317, 318, 319 and 320. If the pain persists in the inner ankle and heel after stimulating all of the stimulation points, the patient should locate the exact origin of the point of pain and stimulate that point.

Again referring to FIGS. 15, 16 and 17 for pain in the top of the foot, the patient should sequentially stimulate points 321, 322, 323, 324, 325, 326 and 327. If the patient still feels pain after stimulating all of the stimulation points for the top of the foot, he or she should locate the exact origin of the point of pain and stimulate that point. For pain in the ball of the foot, the patient should stimulate points 328, 329, 330, 331 and 332 sequentially. If the patient still feels pain in the ball of the foot after stimulating all of the stimulation points, he or she should locate the exact origin of the point of pain and stimulate that point.

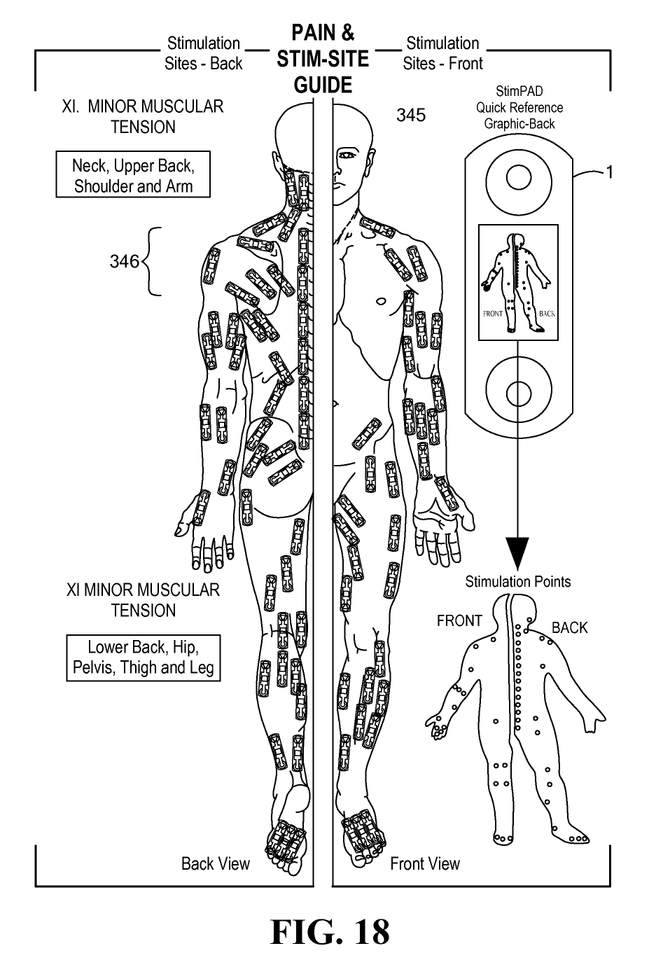

FIG. 18 is an overview of the stimulation points on the front and back of the body. The picture divides a human body into two halves, a front half 345 on the right side and a back half 346 on the left side. The stimulation points identified in the diagram are repeated from FIGS. 3-17. The upper portion of the displayed stimulation points may be used to alleviate neck, upper back, shoulder and arm pain associated with minor muscular tension. The lower portion of the displayed stimulation points may be used to alleviate lower back, hip, pelvis, thigh and leg pain associated with minor muscular tension. In one embodiment of the electronic stimulation apparatus, this picture is placed on the backside of the apparatus 1 to assist the patient with quickly identifying stimulation points on the body.

A variation of the above embodiment includes a sensor incorporated into the apparatus 1, which assists the user in locating the stimulation points and the exact origin of pain. The sensor is a transepithelia potential indicator, which measures frequency across the skin to identify termination of nerve ends within the body. When the sensor passes over the nerve ends, the frequency across the skin changes and the sensor alerts the patient to notify the patient that it has passed the apparatus 1 over a stimulation point.

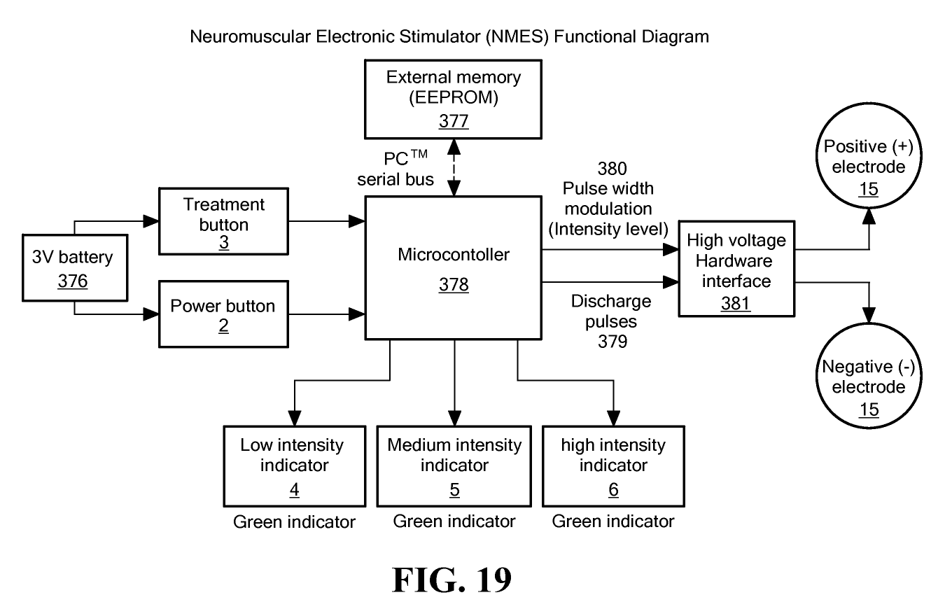

FIG. 19 is a functional diagram of the self-contained electronic stimulation apparatus. The stimulation apparatus contains a 3-volt battery 376. The battery 376 connects to the treatment button 3 and the power button 2. The power button 2 allows a user to turn the apparatus 1 on, off and select an intensity level for treatment. The treatment button 3 activates the electronic stimulation. Both the treatment button 3 and the power button 2 connect to the microcontroller 378. The microcontroller 378 is a PIC 16LC505 consisting of complementary metal-oxide semiconductor (“CMOS”) material. The microcontroller 378 controls the three intensity indicators, the low intensity indicator 4, the medium intensity indicator 5 and the high intensity indicator 6. Also connected to the microcontroller 378 is an external memory 377. The external memory 377 attaches to the microcontroller 378 by a serial bus. One embodiment uses a serial bus sold under the trademark I²C® of Koninklijke Philips Electronics, N.V. The external memory 377 is an electrically erasable programmable read-only memory (“EEPROM”). The memory is called external because it is outside of the microcontroller 378, however the external memory 377 is located inside the apparatus 1. The external memory 377 stores the power level intensity, a password, and keeps a log of the number of complete treatments by using a counter. The password is used to format the apparatus 1 when it is first turned on, to store the level of intensity to off, and to set the counter to zero.

The microcontroller 378 and the external memory 377 work in conjunction with each other so that the power intensity level of the apparatus 1 is always in the off position. Therefore, the apparatus 1 only releases an electronic stimulation while the patient has the treatment button 3 pressed. When the patient ceases depressing the treatment button 3, the apparatus 1 will turn to the off intensity level and cease exerting an electronic stimulation. Thus, the patient controls the duration of the treatment, if less than the full four second treatment is desired. During an electronic stimulation, the microcontroller 378 discharges a particular pulse 379 corresponding to the intensity level chosen by the patient. The microcontroller 378 also outputs a pulse-width modulation or intensity level 380. Both the intensity level 380 data and the discharge pulses 379 are received by the high voltage hardware interface 381. The high voltage hardware interface 381 connects to both the positive and negative electrodes 15.

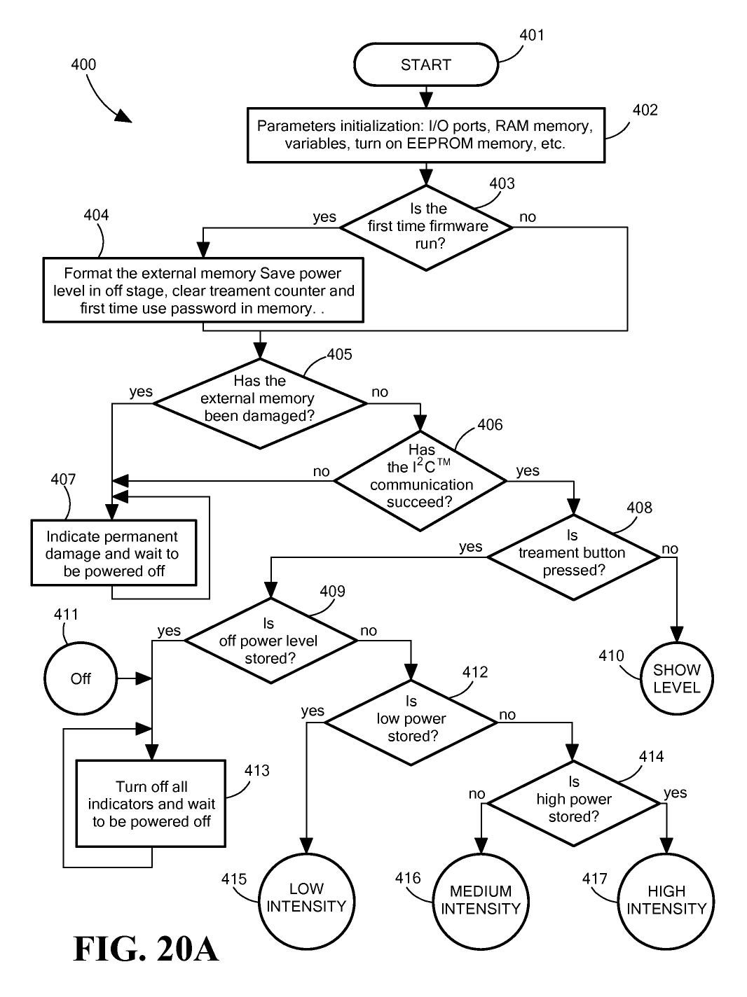

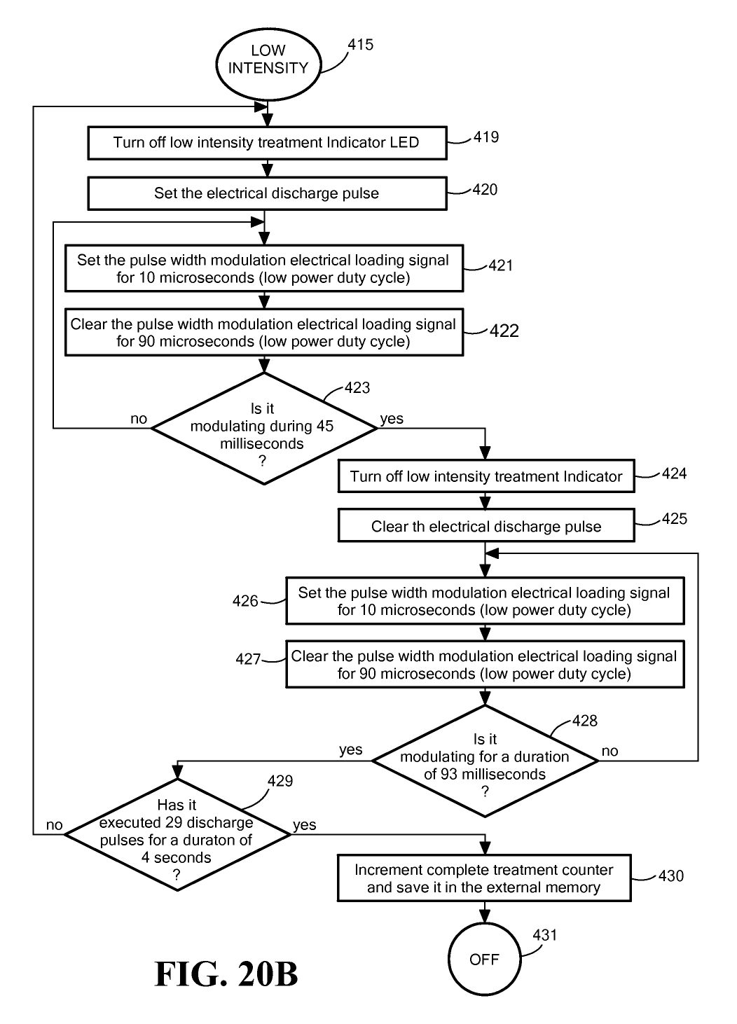

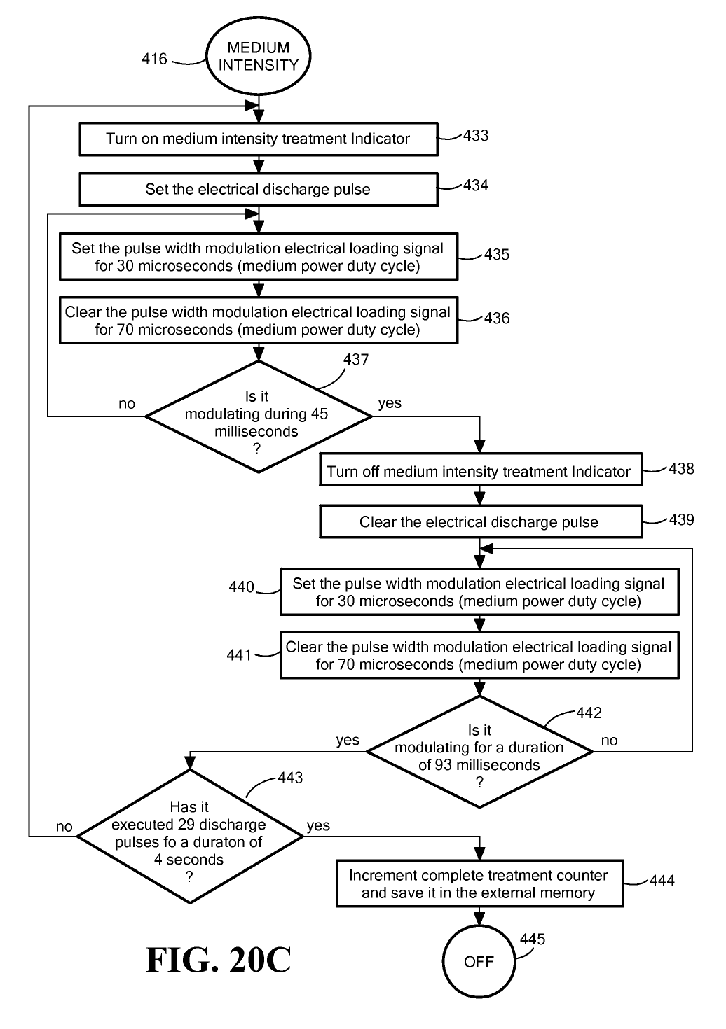

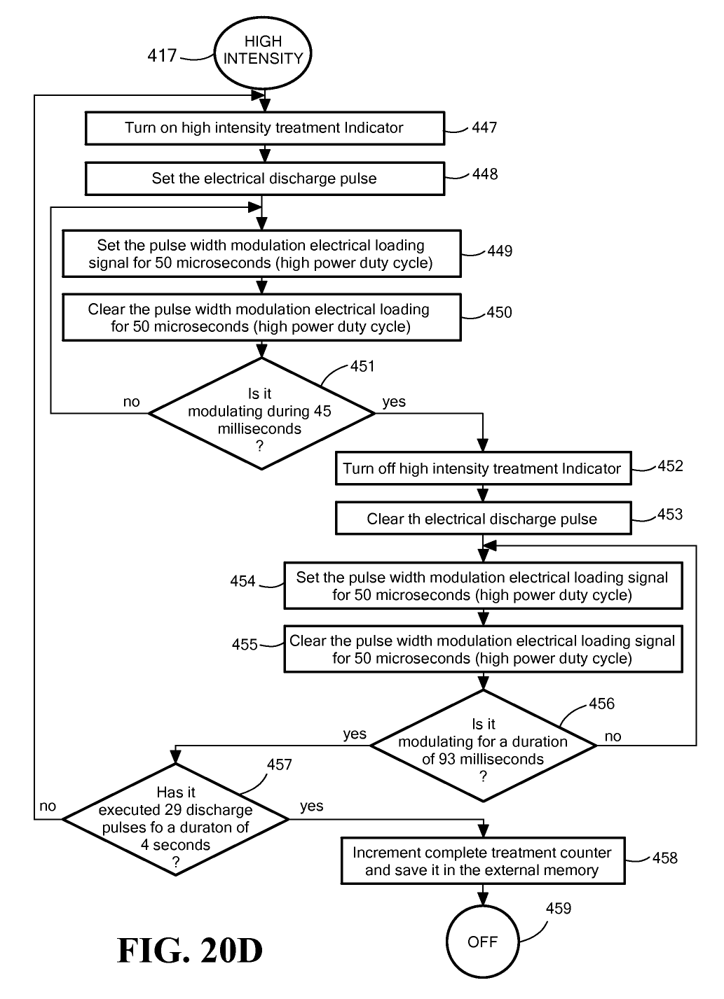

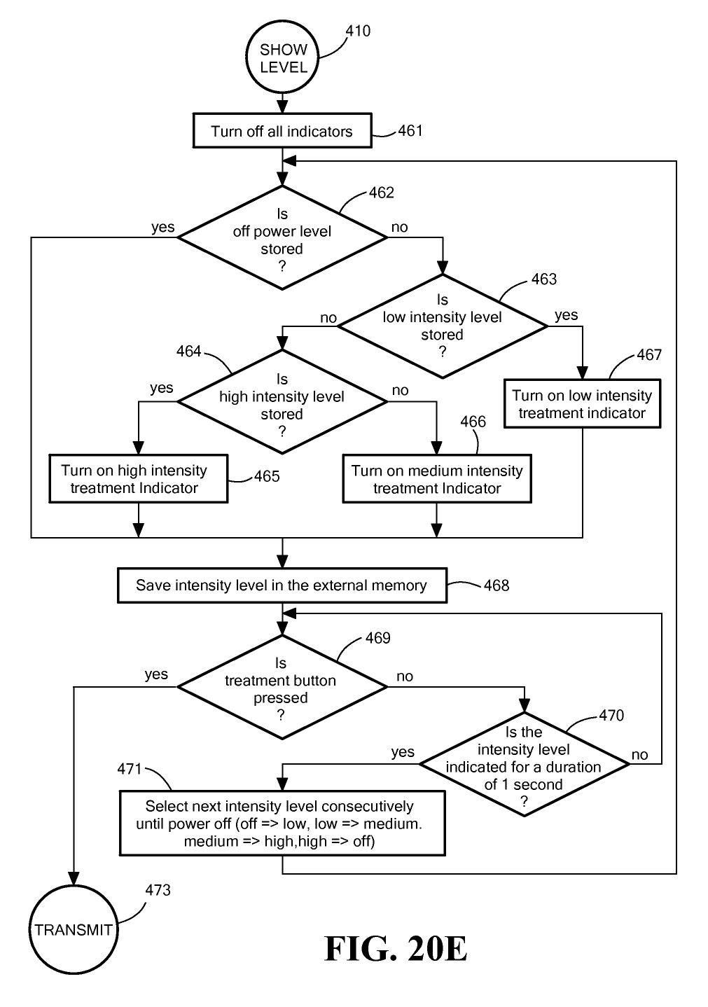

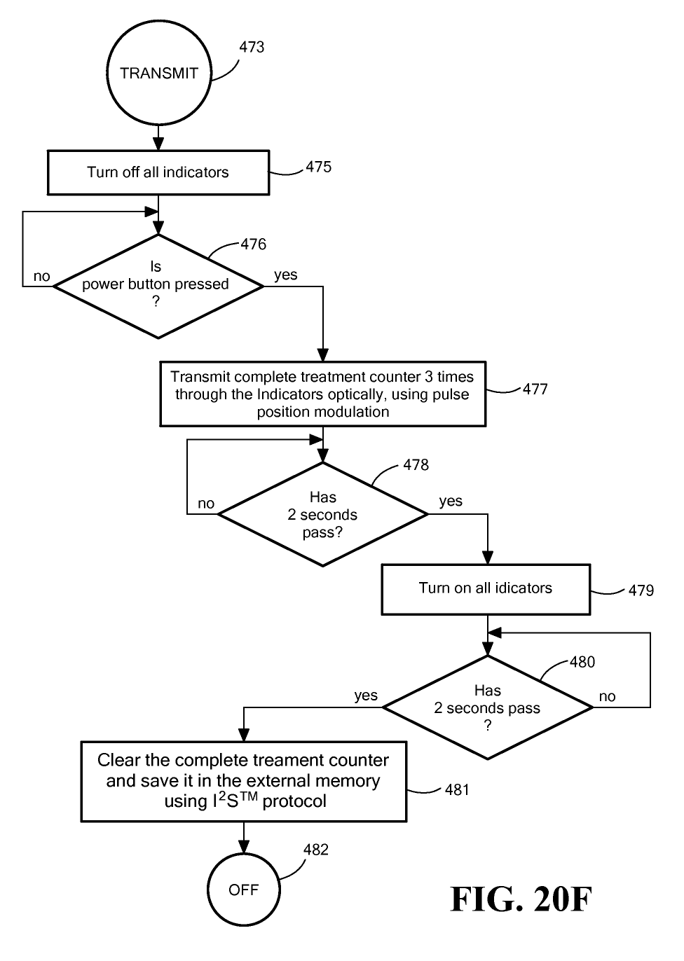

FIGS. 20A-F are logic flowcharts of the self-contained electronic stimulation apparatus software 400. The flowcharts begin at FIG. 20A at the start 401. The first step is that the parameters are initialized 402. For example, some of the parameters that are initialized are the in and out ports, the random access memory, the systems variables, and the external memory is turned on. Next, the system determines if it is the first time that the firmware is run 403. If it is the first time the firmware is run 403, the external memory is formatted, the power level is saved in the off stage, the treatment counter is cleared and the password is entered in the memory 404. This step ensures that the apparatus 1 is always in the off position unless the treatment button 3 is pressed. After step 404 or if it is determined to not be the first time the firmware is run 403, then the system proceeds to determine if the external memory has been damaged 405. If the external memory has not been damaged 405, then the systems checks if the I²C® communication has succeeded 406. The I²C® is a computer language communications protocol between the external memory and the microcontroller. If the external memory has been damaged 405 or if the I²C® communication has not succeeded 405, then the system enters a loop whereby it indicates permanent damage and waits to be powered off 407.

After the system determines that the I²C® communication has succeeded 406, the next step is to detect if the treatment button 3 has been pressed 408. If the treatment button has not been pressed 408, the system moves on to the step of showing the power 410. As continued in FIG. 20E, the indicators 4, 5, and 6 are turned off 461. The system next determines if the off power level has been stored 462. If the off power level has been stored, the system saves the power level in the external memory 468. If the off power level has not been stored 462, the system checks if the low intensity level has been stored 463. If the low intensity level has been stored 463, the system turns on the low intensity treatment indicator 4 and moves on to save the intensity level in the external memory 468. If the low intensity level has not been stored 463, the system determines if the high intensity level has been stored 464. If the high intensity level has been stored, the system turns on the high intensity treatment indicator 6. If the high intensity level has not been stored, the system turns on the medium intensity treatment indicator 5. After determining that the off power level has been stored 462, turning on the high intensity indicator 465, turning on the medium intensity indicator 466 or turning on the low intensity indicator 467; the system will save the intensity level in the external memory 468.

Next, the system determines if the treatment button 3 has been pressed 469. If the treatment button 3 has not been pressed 469, then the system determines that the button being depressed is the power button 2 and the system checks if the intensity level is indicated for a duration of one second 470. If the intensity level is not indicated for a duration of one second 470, the system loops back to check if the treatment button 3 has been pressed 469. If the intensity level has been indicated a duration of one second 470, the next intensity level is selected until it cycles to power off 471. Therefore, the system cycles through the following selections: off, low, medium, high, and off. Once the user releases the treatment button 3, the system loops back to step 462 to determine if the off power level has been stored. If the treatment button 2 has been pressed 469, then the system continues to the transmit step 473.

The transmit step 473 is continued in FIG. 20F. The transmit function allows the self-contained apparatus to communicate via a wireless signal with another device to download usage data to a computer. The transmit function, also called the treatment counter, is designed to be used by the manufacturer of the apparatus to determine the number of times the apparatus has been used and the intensity level that has been used. The treatment counter can also be employed to shut off the device after a predetermined number of uses. The indicators are all turned off 475. Then the system determines if the power button is pressed 476. If the power button is not pressed 476, the system loops up to again determine if the power button has been pressed 476. If the power button has been pressed 476, a complete treatment counter is transmitted three times through the specific indicator 4, 5, or 6 by using a photonic pulse position modulation 477, also known as a wireless modulation technique. Other suitable wireless exchange platforms may also be used to transmit information, such as optical or radio frequency signals. Next, the system determines if two seconds have passed 478. If two seconds have not passed 478, the system loops back to determine again if two seconds have passed 478. If two seconds have passed 478, all indicators are turned on 479. Again, the system determines if two seconds have passed 480. If two seconds have not passed 480, the system loops back to determine again if two seconds have passed 480. If two seconds have passed 480, the complete treatment counter is cleared and saved in the external memory using the I²C® protocol 481. This effectively erases or resets the memory of the number of uses of the device. The system then turns off 482.

The self-contained apparatus transmits a wireless signal to another device in order to download usage data to a computer with the help of specialized software. In one embodiment, the self-contained apparatus transmits a signal to a wireless data transfer device. Software has been created for use by the manufacturer in a WINDOWS® Environment that controls the wireless data transfer device that has been developed for the manufacturer to access the information being transmitted from the stimulation apparatus. The software facilitates uploading and monitoring of warranty data and patient usage and then generates reports based on that criterion. The software and wireless data transfer device may also be used in a clinical setting whereby a clinician may input patient information into a stimulation apparatus for later billing and record usage, in addition to the completed treatment counter information uploaded from the stimulation apparatus. The software may also retrieve variable patient information, generate billing codes, bookkeeping information, mailing addresses, and other pertinent information that was previously input by the clinician for monitoring purposes.

Referring back to FIG. 20A, step 408, if it is determined that the treatment button has been pressed 408, then the system checks if the off power level has been stored 409. If the off power level has been stored 409, then the system turns all indicators off and the system waits to be powered off 413. The system loops continuously back through this step. However, if the off power level has not been stored 409, the system continues on to check if the low intensity level has been stored 412. If the low intensity level has not been stored 412, the system continues on to check if the high intensity level has been stored 414. If the low intensity level has been stored, the system moves to low intensity function 415. If the high intensity level has been stored 414, the system moves to high intensity function 417. If the high intensity level has not been stored 441, the system moves to medium intensity function 416.

If the low intensity level has been stored 412, the system moves to low intensity function 415. This is continued on FIG. 20B, where the system turns on the low intensity treatment indicator 419, sets the electrical discharge pulse 420, sets the pulse width modulation electrical loading signal for ten (10) microseconds 421 and clears the pulse-width modulation electrical loading signal for ninety (90) microseconds 422. This process creates the low intensity duty cycle of approximately 9-14%. Next the system checks if it is modulating during forty-five (45) milliseconds 423. If the system is not modulating during forty-five (45) milliseconds 423, then the system loops back and sets the pulse-width modulation electrical loading signal for ten (10) microseconds 421 and clears the pulse-width modulation electrical loading signal for ninety (90) microseconds 422. If the system is modulating during forty-five (45) milliseconds 423, then the system turns off the low intensity indicator 4, clears the electrical discharge pulse 425, sets the pulse-width modulation electrical loading signal for ten (10) microseconds 426 and clears the pulse-width modulation electrical loading signal for ninety (90) microseconds 427.

The following step determines if the system is modulating for a duration of ninety-three (93) milliseconds 428. If the system is not modulating for a duration of ninety-three (93) milliseconds 428, it repeats steps 426 and 427. Thus, for forty-five (45) milliseconds, the pulse is output through the electrodes and for ninety-three (93) milliseconds, no pulse is output through the electrodes. If it is modulating for a duration of ninety-three (93) milliseconds, the system determines if it has executed twenty-nine (29) discharge pulses for a duration of four seconds 429. If it has not executed twenty-nine (29) discharge pulses for a duration of four seconds 429, the system loops back to step 419. If the system has executed twenty-nine (29) discharge pulses for a duration of four seconds 429, the system increments a complete treatment counter and saves it in the external memory 430. The system then turns off 431.

Referring back to FIG. 20A, if the high intensity level is not stored 441, the system moves to medium intensity function 416. This process is continued in FIG. 20C, where the system turns on the medium intensity treatment indicator 433, sets the electrical discharge pulse 434, sets the pulse-width modulation electrical loading signal for thirty (30) microseconds 435 and clears the pulse-width modulation electrical loading signal for seventy (70) microseconds 436. This process creates the medium intensity duty cycle of approximately 26-31%. Next the system checks if it is modulating during forty-five (45) milliseconds 237. If the system is not modulating during forty-five (45) milliseconds 437, then the system loops back and sets the pulse-width modulation electrical loading signal for thirty (30) microseconds 435 and clears the pulse width modulation electrical loading signal for seventy (70) microseconds 436. If the system is modulating during forty-five (45) milliseconds 437, then the system turns off the medium intensity indicator 438, clears the electrical discharge pulse 439, sets the pulse-width modulation electrical loading signal for thirty (30) microseconds 440 and clears the pulse-width modulation electrical loading signal for seventy (70) microseconds 441.

The following step determines if the system is modulating for a duration of ninety-three (93) milliseconds 442. If the system is not modulating for a duration of ninety-three (93) milliseconds 442, it repeats steps 440 and 441. Thus, for forty-five (45) milliseconds, the pulse is output through the electrodes and for ninety-three (93) milliseconds, no pulse is output through the electrodes. If it is modulating for a duration of ninety-three (93) milliseconds, the system determines if it has executed twenty-nine (29) discharge pulses for a duration of four (4) seconds 443. If it has not executed twenty-nine (29) discharge pulses for a duration of four (4) seconds 443, the system loops back to step 433. If the system has executed twenty-nine (29) discharge pulses for a duration of four (4) seconds 443, the system increments a complete treatment counter and saves it in the external memory 444. The system then turns off 445.

Referring back to FIG. 20A, if the high intensity level is stored 414, the system moves to high intensity function 417. This process is continued in FIG. 20D, where the system turns on the high intensity treatment indicator 447, sets the electrical discharge pulse 448, sets the pulse-width modulation electrical loading signal for fifty (50) microseconds 449 and clears the pulse-width modulation electrical loading signal for fifty (50) microseconds 450. This process creates the high intensity duty cycle of approximately 47-53%. Next the system checks if it is modulating during forty-five (45) milliseconds 451. If the system is not modulating during forty-five (45) milliseconds 451, then the system loops back and sets the pulse-width modulation electrical loading signal for fifty (50) microseconds 449 and clears the pulse-width modulation electrical loading signal for fifty (50) microseconds 450. If the system is modulating during forty-five (45) milliseconds 451, then the system turns off the high intensity indicator 452, clears the electrical discharge pulse 453, sets the pulse-width modulation electrical loading signal for fifty (50) microseconds 454 and clears the pulse-width modulation electrical loading signal for fifty (50) microseconds 455.

The following step determines if the system is modulating for a duration of ninety-three (93) milliseconds 456. If the system is not modulating for a duration of ninety-three (93) milliseconds 456, it repeats steps 454 and 455. Thus, for forty-five (45) milliseconds, the pulse is output through the electrodes and for ninety-three (93) milliseconds, no pulse is output through the electrodes. If it is modulating for a duration of ninety-three (93) milliseconds 456, the system determines if it has executed twenty-nine (29) discharge pulses for a duration of four (4) seconds 457. If it has not executed twenty-nine (29) discharge pulses for a duration of four (4) seconds 457, the system loops back and enters the system at step 447. If the system has executed twenty-nine (29) discharge pulses for a duration of four (4) seconds 457, the system increments a complete treatment counter and saves it in the external memory 458. The system then turns off 459.

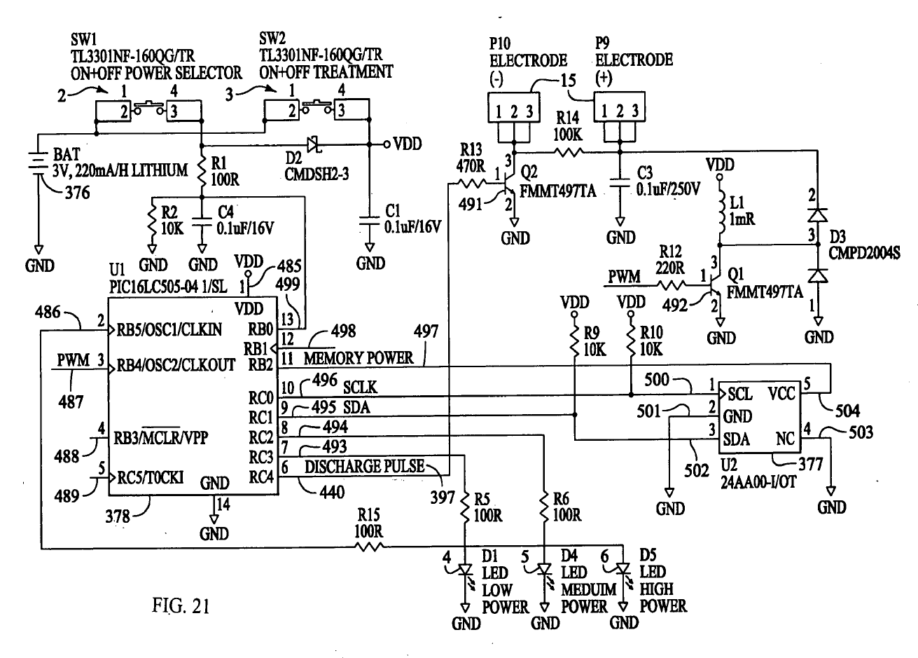

FIG. 21 depicts one example of an embodiment of a circuit found in the self-contained electronic musculoskeletal stimulation apparatus 1. The circuit displays two switches; the power button 2 and the treatment button 3, both connected to the microcontroller 378 by a key detection pathway. The microcontroller 378 connects to several pathways. Connection 485 supplies power to the microcontroller 378 from the battery 376 when a button is pressed. Connection 486 attaches to the high intensity indicator 6. Connection 487 is the pulse-width modulation input. Connection 488 and 489 are not used. Connection 490 is the discharge pulse 379 and is the same for each intensity level, which is, in the preferred embodiment, approximately seven hertz for four seconds. Depending upon the particular embodiment and uses of the apparatus, the frequency may range from 0.1 to 4000 hertz. The discharge pulse 379 leads to the capacitors 491 and 492 then to the positive and negative electrodes 15 to output an electrical charge. The capacitors 491 and 492 function to store the voltage output and release the discharge voltage 379. For example, when low intensity is selected, the capacitors 491 and 492 receive voltage for ten microseconds, then do not receive voltage for the remaining ninety microseconds. This process creates the low intensity duty cycle of approximately 9-14%. Connection 493 from the microcontroller 378 attaches to the low level indicator 4 and connection 494 attaches to the medium intensity indicator 5. Connection 495 provides serial data to the external memory 377 and connection 496 provides serial clock information to the external memory 377. Connection 497 provides power to the external memory 377. Connection 498 is not used. Connection 499 detects whether the power button 2 or treatment button 3 has been pressed.

The external memory also has several input and output connections. Connection 500 is the serial clock input from the microcontroller 378. Connection 501 is a ground connection. Connection 502 the serial data input from the microcontroller 378. Connection 503 is a ground connection. Connection 504 is power input from the microcontroller 378.

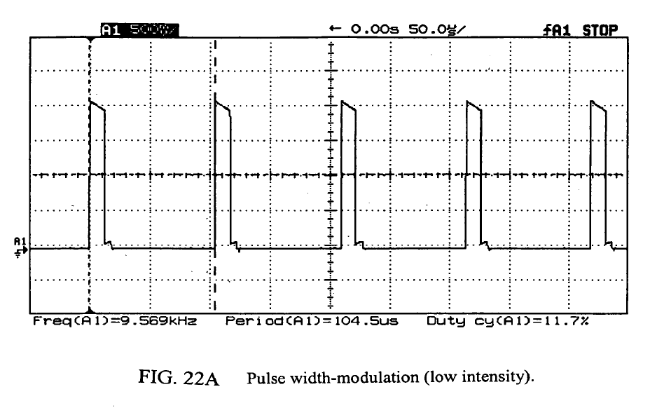

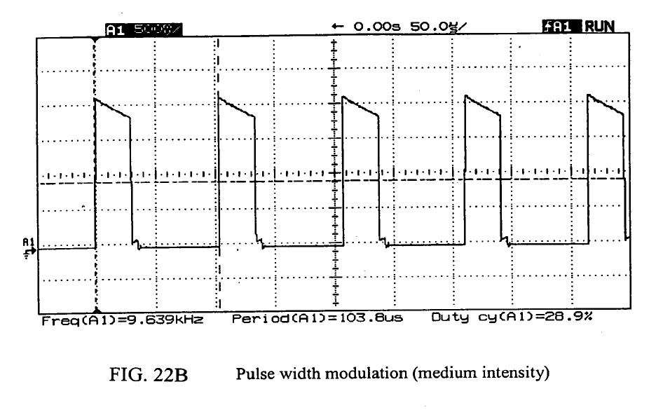

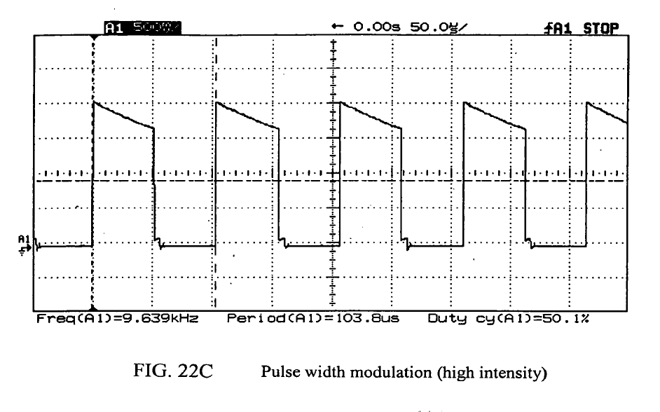

FIGS. 22A-C show three different pulse-width modulation technique graphs. These graphs display the variety of pulse widths and respective duty cycles that may be used with the electronic stimulation apparatus. A pulse-width modulation is a type of pulse-time modulation in which pulse duration is varied by the modulation. The duty cycle represents the ratio of on-time to idle-time during the operation of the stimulation apparatus. The pulse-width can range between 0.01 microseconds to 50 milliseconds depending upon the particular apparatus and use. In the preferred embodiment, the pulse-width is approximately 45 milliseconds. FIG. 22A is a graph of a pulse-width modulation used for the low intensity level. The low intensity pulse-width modulation has a duty cycle of approximately 11.7% and the period of the pulse is 104.5 microseconds. FIG. 22B is a graph of a pulse-width modulation used with the medium intensity level. The medium intensity pulse-width modulation has a duty cycle of approximately 28.9% and the period of the pulse is 103.8 microseconds. FIG. 22C is a graph of a pulse-width modulation used for the high intensity level. The high intensity pulse-width modulation uses a duty cycle of approximately 50.1% and the period of the pulse is 103.8 microseconds.

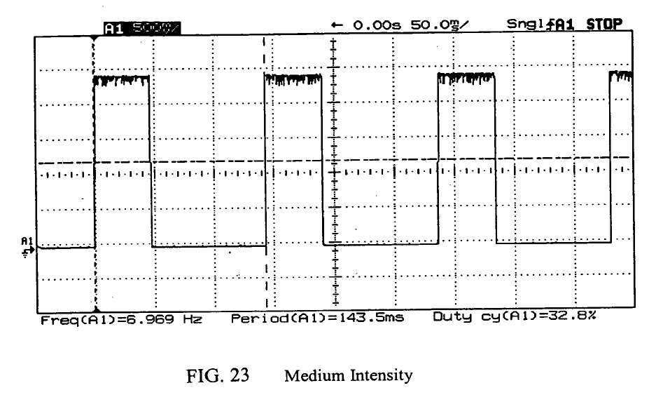

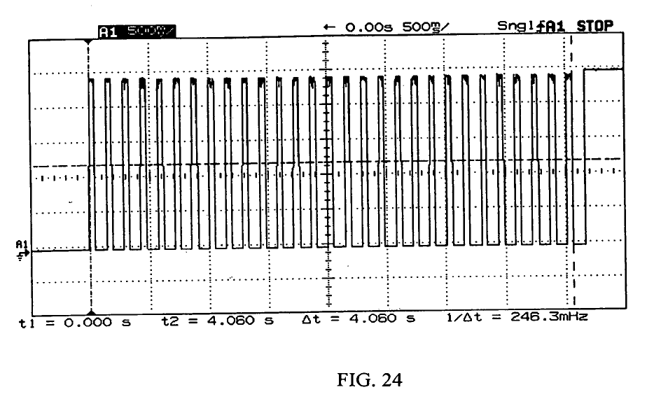

FIG. 23 is a graph of the discharge pulse waveform. The waveform is a square waveform meaning it has an alternating or pulsating current or voltage whose wave shape is square. The pulse shown is a medium intensity level and the duty cycle of the shown pulse is 32.8%. FIG. 24 shows the total discharge pulses of approximately 29 pulses in four seconds. The amount of total discharge pulses is similar for each level of intensity.

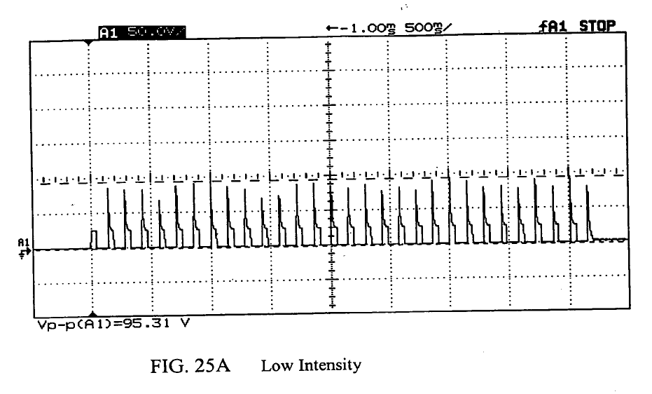

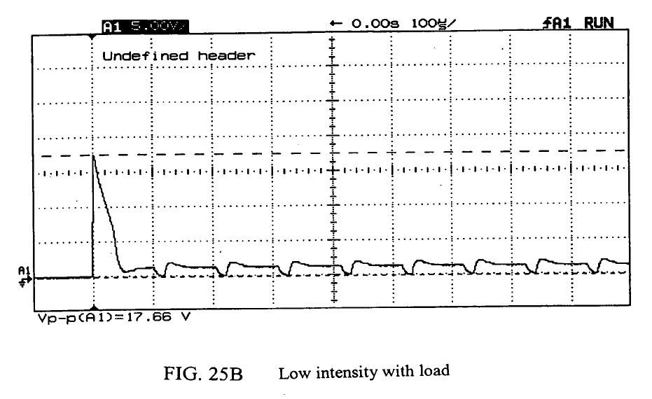

FIGS. 25A and B, 26A and B, and 27A and B represent data collected from one embodiment of the invention using various intensity level selections. It should be understood that the data in no way limits the invention and is shown only to provide examples of data from an embodiment of the invention. The particular embodiment employed to collect the data in FIGS. 25A and B, 26A and B and 27A and B was a self-contained electronic stimulation apparatus as shown in FIGS. 1 and 2. FIG. 25A is a graph of the total low intensity electrical pulses generated from the positive electrode during treatment with a stimulation apparatus. The stimulation apparatus produces thirty pulses within approximately four seconds. FIG. 25B graphs the low intensity electrical pulse generated from the positive electrode of a stimulation apparatus, with a fifty (50) ohms load resistance. The fifty (50) ohms load represents the approximate equivalent impedance of the human body. When the resistance is applied, the voltage from the low intensity is 17.66 volts.

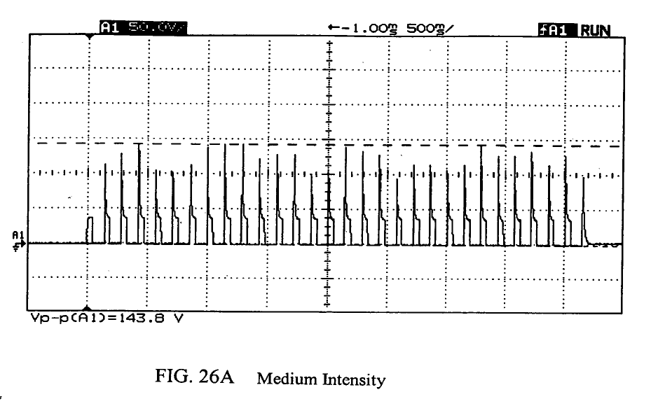

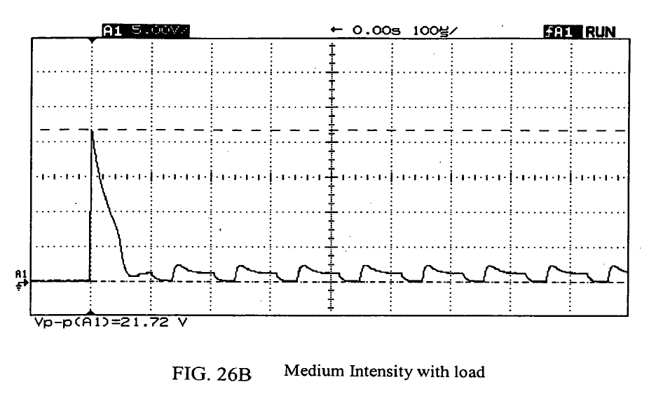

FIG. 26A is a graph of the total medium intensity electrical pulses generated during treatment with a stimulation apparatus. The stimulation apparatus produces thirty (30) pulses within approximately four seconds. FIG. 26B graphs the medium intensity electrical pulse generated from the stimulation apparatus with fifty (50) ohms load resistance. The fifty (50) ohms load resistance represents the average impedance of the human body. When the resistance is applied, the voltage from the medium intensity is 21.72 volts.

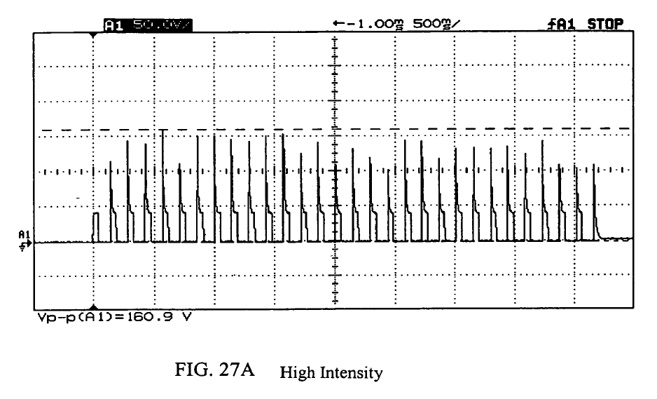

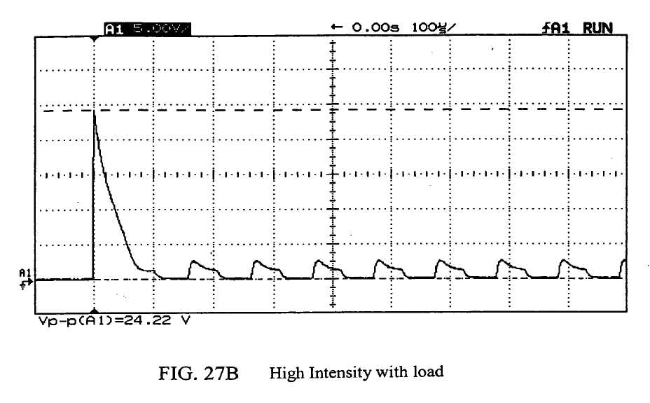

FIG. 27A is a graph of the total number of high intensity electrical pulses generated from the positive electrode of the stimulation apparatus. The stimulation apparatus produces thirty (30) pulses within approximately four seconds. FIG. 27B graphs the high intensity electrical pulse generated from the stimulation apparatus with a fifty (50) ohms load resistance. The fifty (50) ohms load resistance represents the average impedance of the human body. When the resistance is applied, the voltage from the medium intensity is 24.22 volts.

Accordingly, it will be understood that the preferred embodiment of the present invention has been disclosed by way of example and that other modifications and alterations may occur to those skilled in the art without departing from the scope and spirit of the appended claims.

1-47. (canceled)

48. A method of applying pain relieving electronic stimulation to a body, comprising:
affixing a self-contained electronic musculoskeletal stimulation apparatus to a first predetermined stimulation point on the body;
selecting an intensity of the electronic stimulation, wherein the selection is identified by at least one indicator;
activating the electronic stimulation, the electronic stimulation being controlled at least in part by a microprocessor allowing for at least one of multiple frequencies or pulse variations;
removing the apparatus from the first predetermined stimulation point;
affixing the apparatus to a second predetermined stimulation point on the body;
selecting an intensity of the electronic stimulation; and
activating the electronic stimulation.

49. The method of claim 18, further comprising detecting one of a stimulation point and a pain location on the body with the apparatus.

50. The method of claim 49, wherein the detection includes measuring a frequency across a skin portion of the body.

51. The method of claim 48, further comprising electronically storing information about at least one of the selection of the intensity and activation of the electronic stimulation.

52. The method of claim 48, further comprising positioning at least one electrogel pad between the apparatus and the body.

53. The method of claim 52, wherein the at least one electrogel pad includes a medicinal compound.

54. The method of claim 52, wherein the at least one electrogel pad includes an adhesive for anchoring the apparatus to the body.

55. The method of claim 48, further comprising selecting a duration of time for the electronic stimulation.

56. The method of claim 48, further comprising defining a plurality of sequential stimulation points on the body, wherein the plurality of sequential stimulation points includes the first and second predetermined stimulation point.

57. The method of claim 48, further comprising:
moving a portion of the body where the pain is occurring;
determining if pain is still occurring;
affixing the apparatus directly on a location of the pain; and
activating the electronic stimulation.

58. The method of claim 48, wherein the stimulation apparatus includes a control circuit connected to two or more electrodes, the control circuit and the electrodes being contained within a singular housing.

59. The method of claim 58, wherein the electronic stimulation is provided at least in part by applying a constant current to the electrodes and generating a voltage adjustable between a plurality of intensities.

60. The method of claim 59, wherein the stimulation apparatus has an adjustable voltage intensity ranging from approximately 90 volts to approximately 180 volts.

61. The method of claim 59, further comprising applying the current with a burst of approximately 30 pulses for approximately a four second duration.

62. A method of applying pain relieving electronic stimulation to a body, comprising:
identifying a location of pain on the body;
defining a plurality of sequential stimulation points on the body;
affixing a self-contained electronic musculoskeletal stimulation apparatus to the body at a first stimulation point of the defined plurality of sequential stimulation points;
selecting an intensity of the electronic stimulation;
activating the electronic stimulation,
removing the apparatus from the first predetermined stimulation point;
affixing the apparatus to the body at a second stimulation point of the defined plurality of sequential stimulation points, wherein the second stimulation point is closer to the identified location of pain than the first stimulation point;
selecting an intensity of the electronic stimulation; and
activating the electronic stimulation.

63. The method of claim 62, the electronic stimulation being controlled at least in part by a microprocessor allowing for at least one of multiple frequencies or pulse variations;

64. The method of claim 62, wherein the at least one indicator visually indicates the selected intensity of the electronic stimulation.

65. The method of claim 62, wherein the stimulation apparatus includes an indicator indicating that electronic stimulation is activated.

66. A method of applying pain relieving electronic stimulation to a body, comprising:
identifying a location of pain on the body;
defining a plurality of sequential stimulation points on the body;
affixing a self-contained electronic musculoskeletal stimulation apparatus to the body at a first stimulation point of the defined plurality of sequential stimulation points, the apparatus including a control circuit connected to two or more electrodes contained in a singular housing;
selecting an intensity of the electronic stimulation, wherein the apparatus visually indicates the selected intensity;
activating the electronic stimulation, the electronic stimulation being controlled at least in part by a microprocessor allowing for at least one of multiple frequencies or pulse variations;
removing the apparatus from the first predetermined stimulation point;
affixing the apparatus to the body at a second stimulation point of the defined plurality of sequential stimulation points, wherein the second stimulation point is closer to the identified location of pain than the first stimulation point;
selecting an intensity of the electronic stimulation; and
activating the electronic stimulation.

67. The method of claim 66, further comprising detecting one of a stimulation point and a pain location on the body with the apparatus.
